(19) United States
(12) Patent Application Publication
(10) Pub. No.: US 2009/0156975 A1
(43) Pub. Date: Jun. 18, 2009

(54) Robust System and Methods for Blood Access

(76) Inventors: Mark Ries Robinson, Albuquerque, NM (US); Mike Borrello, Carlsbad, CA (US); Dan Welsh, Encinitas, CA (US)

Correspondence Address: V. Gerald Grafe, esq., P.O. Box 2689, Corrales, NM, 87048, US

(21) Appl. No.: 12/325,243

(22) Filed: Nov. 30, 2008

Related U.S. Application Data
(60) Provisional application No. 61/044,004, filed on Apr. 10, 2008.
(60) Provisional application No. 60/991,373, filed on Nov. 30, 2007.
(60) Provisional application No. 60/991,447, filed on Nov. 30, 2007.
(60) Provisional application No. 60/992,037, filed on Dec. 3, 2007.

(51) Int. Cl. A61M 1/14; B01D 65/02

(52) U.S. Cl. 604/4.01; 210/636

(57) ABSTRACT

Embodiments of the present invention provide robust systems for the removal and subsequent infusion of blood for measurement purposes, and embodiments of the present invention provide methods of operating such systems and providing capabilities such as predicting and avoiding occlusions and/or bubbles, managing occlusions and/or bubbles if they occur, automatic cleaning of the blood access system, and determining and managing the patency of the blood access site. Such operational challenges can occur during any of several phases of operation of a blood access system. Embodiments of the present invention can effectively incorporate a variety of inputs for the identification of trends consistent with present or pending occlusions. An embodiment of the present invention can be aware of the stage of operation, e.g., withdrawal, infusion, or cleaning, and the prior performance of the system. Embodiments of the present invention can have the ability to identify the location of the problem so that effective procedures can be used to resolve the problem.

### CROSSREFERENCE TO RELATED APPLICATIONS

This application claims the benefit of U.S. provisional application 61/044,004, filed Apr. 10, 2008, and of U.S. provisional application 60/991,373, filed Nov. 30, 2007; and of U.S. Provisional Application No. 60/991,447, filed Nov. 30, 2007; and of U.S. Provisional Application No. 60/992,037, filed Dec. 3, 2007; each of which is incorporated herein by reference.

### FIELD OF THE INVENTION

The present invention relates to the field of accessing blood, and more specifically to methods and apparatuses that remove blood from a patient for treatment or analysis and return at least a portion of the blood to the patient.

### BACKGROUND

In recent years, the frequent monitoring of blood parameters has become much more common. For example blood glucose is commonly monitored for the implementation of tight glycemic control protocols, and lactate is monitored for the general perfusion assessment. The adoption of care practices that require frequent blood monitoring have necessitated the development of systems that enable blood to be obtained on a regular basis.

In general terms such a blood access system is capable of removing blood from the patient and presenting the blood to a measurement and/or processing system. The measurement system can comprise various measurement technologies, including without limitation ion specific electrodes, optical measurements, plasma separation techniques, ultra filtration, etc. Occlusions in such blood access systems can lead to undesirable outcomes, so it can be important that the system be able to determine when an occlusion has occurred such that a care provider can assess and manage the situation. In addition to the loss of measurement information, rapid detection of occlusions is important since stagnant blood in the blood access system can clot over time and the access site can be lost for future measurements.

Via Medical developed a blood access system that withdrew blood from the body for measurement of glucose and other parameters by ion specific electrodes. The system detected occlusion by having an allowable time for procurement of the sample. The system knew the start time of the withdrawal and was able to sense the arrival of the blood. If the blood did not arrive within a fixed period of time then the system would alarm.

CHF Solutions has developed a system for ultra filtration that uses pressure thresholds for determination of an occlusion. The system is based upon a pressure measurement made on the access line used for blood removal and a second pressure measurement used on the infusion line. The system has two thresholds that are used for the determination of an occlusion.

Current blood access systems do not provide an ability to monitor or assess the patency (or viability) of the blood access site. Current systems generally operate until an occlusion occurs, trigger an alarm, and then stop operations until medical personnel clear the occlusion. Although existing blood access systems do not evaluate site patency, medical personnel routinely desire to evaluate the patency of a blood access site.

Medical personnel manually determine the viability of an access site by drawing and infusing blood and/or saline through the catheter using a syringe. They perform this check both at the time of catheter insertion and at later times when the patency of the site is in question. Equally importantly, medical personnel clear small occlusion from within catheters by generating high pressure/high rate flows using a syringe directly attached to the catheter. The pressure and flows generated by medical personnel frequently exceed the safe limits that automated systems are allowed to generate. Blood access systems typically operate in one of two fundamental modes: flow control or pressure control. During flow control, the pumping system is targeting a predetermined flow rate and uses the pumping system generates whatever pressure is required to reach this flow target. For safety, the maximum infusion pressure and minimum withdrawal pressure are normally limited. If the system reaches either of these limits during pumping operations, an occlusion is declared and pumping stops. When a blood access system operates in pressure control mode, the pumps turn at whatever rate is required to reach a predetermined pressure value. The pressure value is selected to achieve withdrawal or infusion during a pumping operation. If the desired volume of fluid is not pumped during a preset period of time, the system declares an occlusion and stops pumping.

However, existing blood access systems still do not provide sufficiently robust operation to operate with the reliability desired, such as predicting and avoiding occlusions and/or bubbles, managing occlusions and/or bubbles if they occur, automatic cleaning of the blood access system, and determining and managing the patency of the blood access site.

### BRIEF DESCRIPTION OF THE DRAWINGS

FIG. 1 is a schematic illustration of an example blood access system according to the present invention.

FIG. 2 is a schematic illustration of example pulse commands for the blood and flush lines.

FIG. 3 is a schematic illustration of flow in an example rapid blood withdrawal.

FIG. 3 is a schematic illustration of pressure in an example rapid blood withdrawal.

FIG. 5 is a schematic illustration of commanded flow during an example infusion stage.

FIG. 6 is a schematic illustration of actual flow at a catheter during an example infusion stage.

FIG. 7 is a schematic illustration of commanded flow during an example third infusion stage.

FIG. 8 is a schematic illustration of sensor set flow in blood and flush lines during a scrub stage.

FIG. 9 is a schematic illustration of actual flow at a catheter during a scrub stage.

FIG. 10 is a schematic illustration of sensor set flow in blood and flush lines during a scrub stage using balanced control.

FIG. 11 is a schematic illustration of actual flow at a catheter during a scrub stage using balanced control.

FIG. 12 is a schematic illustration of blood and flush pump commands during an example recirculation stage of cleaning.

FIG. 13 is a schematic illustration of flow commands to the blood and flush pumps during an example catheter flush stage.

FIG. 14 is a schematic illustration of actual flow at the catheter tip during an example catheter flush stage.

FIGS. 15 and 16 comprise decision flow charts that can be used for occlusion detection and management as well as air bubble detection and management in embodiments of the present invention.

FIG. 17 is a schematic illustration of a blood access site patency evaluation implemented on blood draws performed on a swine.

FIG. 18 is a schematic illustration of two pressure measurements during a normal draw and infusion cycle.

FIG. 19 is a schematic illustration of the state of the pressures at an advanced time where blood has aggregated in the catheter.

FIGS. 20 and 21 comprise schematic illustrations of examples of phase space progression comparing flows-pressure and pressure-pressure relations respectively from normal operation to near full occlusion.

FIG. 22 is a schematic illustration of an automated blood access system that contains two fluid bags providing for at least two different calibration points.

FIG. 24 is a schematic illustration of an example implementation of a blood access system with a single level sensor calibration device.

FIG. 26 is a schematic illustration of an example blood access system embodiment.

FIG. 27 is a schematic illustration of an example blood access system embodiment.

FIG. 28 is a schematic illustration of an example blood access system embodiment which also contains the ability to infuse therapeutic amounts of glucose for the effective implementation of tight glycemic control protocols.

FIG. 29 is a schematic illustration of a layout of the functional elements of an example embodiment of an automated device for analyzing blood parameters.

FIG. 30 is a schematic illustration of the layout of the functional elements and workflow of an example embodiment of an automated blood analysis device.

FIG. 31 is a schematic illustration of the layout of the functional elements and workflow of an example embodiment of a blood analysis device.

FIGS. 32A and 32B illustrate an example embodiment of a blood access and measurement system.

FIG. 33 is a schematic illustration of an example blood access system that can be used for automated blood glucose monitoring

### DESCRIPTION OF THE INVENTION

Embodiments of the present invention provide robust systems for the removal and subsequent infusion of blood for measurement purposes, and embodiments of the present invention provide methods of operating such systems and providing capabilities such as predicting and avoiding occlusions and/or bubbles, managing occlusions and/or bubbles if they occur, automatic cleaning of the blood access system, and determining and managing the patency of the blood access site. Such operational challenges can occur during any of several phases of operation of a blood access system. Embodiments of the present invention can effectively incorporate a variety of inputs for the identification of trends consistent with present or pending occlusions. An embodiment of the present invention can be aware of the stage of operation, e.g., withdrawal, infusion, or cleaning, and the prior performance of the system. Embodiments of the present invention can have the ability to identify the location of the problem so that effective procedures can be used to resolve the problem. The following description first describes aspects of the present invention in the context of a particular class of blood access system and particular measurement technology. The invention is not necessarily limited to that class of blood access system or that type of measurement technology; descriptions of other types of blood access systems that are suitable for use with the present invention are also provided. Also, the invention is not necessarily limited to systems incorporating all of the features or elements described herein; subsets of the features and elements can provide useful systems for many applications, and can be incorporated into other systems to improve the performance of such other systems.

In the operation of a blood access system, operational stoppages or disruptions can result from one or more of the following:
1) Occlusions in the catheter due to formation of a clot that restricts the flow of blood or saline in the system. These types of occlusions can occur rapidly and can also occur over several withdrawal cycles.
2) Occlusions in the connection between the measurement circuit and the catheter at the Luer junction due to formation of a clot that restricts the flow of blood or saline in the system. These types of occlusions can occur rapidly and can also occur over several withdrawal cycles.
3) Occlusions due to the location of a catheter tip in the blood vessel or a “kinking” of an access line. These types of occlusions are typically referred to as positional occlusions.
4) Air bubble in the access system that requires stoppage to preclude the air bubble being infused into the patient.
5) Waste bag becomes full.
6) Saline bag used for infusion becomes empty.
7) Occlusions can occur at variety of other locations within the circuit and for a multitude of reasons.

Embodiments of the present invention can use some or all of the following information for detection and management of occlusions and/or air bubbles (and to provide other features and capabilities as described elsewhere herein):
1) Pressure thresholds based upon the stage of operation.
2) Relationship of pressure between two pressure or flow sensors responsive to different portions of the system.
3) The time history of the pressure or flow relationship between different two or more different portions of the system.
4) The time history of pressure or flow measurements (also know as trend changes).
5) Dissipation of pressure within the circuit (the pressure change between the withdrawal and sample stages).
6) Time to complete a stage or time to complete stages of operation.
7) Pressure trends between subsequent withdrawal stages of operation.
8) Flow rates.

In addition to occlusions, embodiments of the present invention also can recognize an air bubble in the line and prevent infusion. The effective detection and subsequent management of air bubbles can facilitate reliable operation of a blood access system. Air bubbles can occur due to out gassing of saline when subjected to negative pressure, from a variety of connection points, and from a priming sequence. It can be desirable for the system to detect the bubble, clear the bubble and resume normal operation.

### EXAMPLE EMBODIMENT

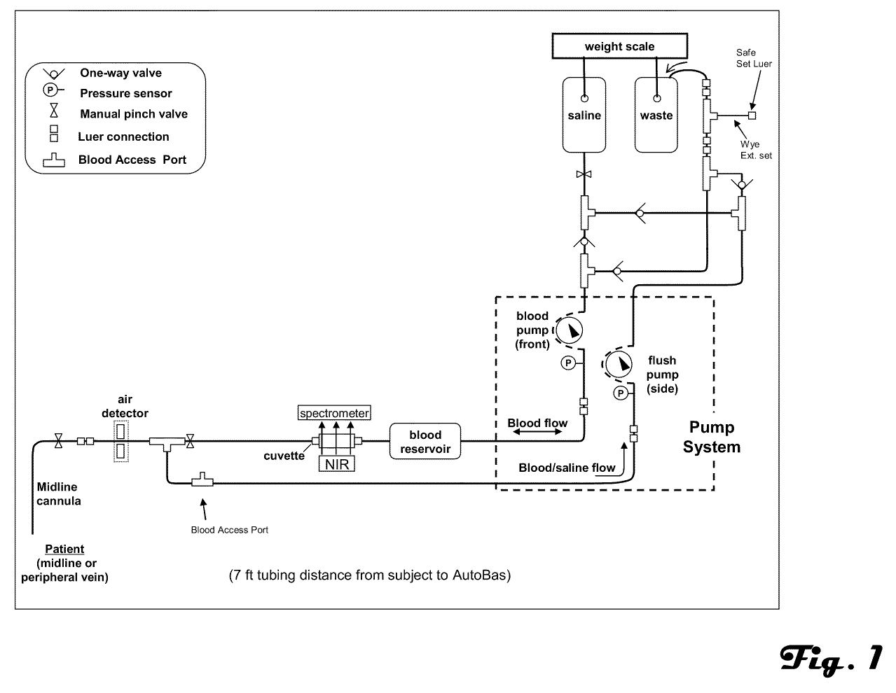

FIG. 1 is a schematic illustration of an example blood access system according to the present invention. Various details are presented in the description of the example system; the present invention contemplates other arrangements, and can operate with more or fewer components than those used in the example illustration. An example blood access system according to the present invention can deliver an undiluted blood sample to an optical measurement system at a distance of up to about 7 feet from the patient. The system can initiate a blood draw (e.g., at the request of a user or under an automated protocol), then pull the blood from the patient and through an optical cuvette for glucose measurement, and then return at least some of the blood to the patient. Conserving blood by returning the sample requires the use and maintenance of a sterile, closed tubing set through which the blood sample is conveyed between the access point and the glucose sensor. In addition to maintaining sterility, the system should also address potential dilution of the blood sample with saline; minimize blood loss and fluid infusion; and clean the fluid circuit to discourage cellular aggregation.

Dilution of the Blood Sample

For descriptive purposes, consider that the sensor set is completely primed with a maintenance fluid, such as saline, to effect non-compliant movement of the blood. As the blood is drawn from the patient through the tubing, the blood—saline interface becomes smeared by laminar flow effects and mixing. The size of this interface between undiluted blood and saline becomes larger as the draw continues. Since many glucose measurement systems cannot tolerate significant dilution, blood can be drawn past the glucose sensor and collected in a tubing reservoir until an undiluted blood sample is presented at the sensor.

Minimization of Blood Loss and Fluids Infusion

The example blood access system provides a balance so that blood loss is minimized while fluid overload of the patient does not occur. In general terms the limit of fluid infusion can be set at approximately 10% of a typical fluid maintenance rate. This involves a careful determination of infused volume to compensate for blood-saline mixing, and the use of specific fluid flow rates and patterns that facilitate cleaning of the conduit during the phases of blood infusion and cleaning. With respect to blood loss, the general desire is to minimize blood loss to the extent possible.

Maintenance of Patency and Minimization of Cellular Aggregation

Extracorporeal blood tends to adhere to foreign surfaces and over a period of a few minutes solidifies. Treating with anticoagulants can reduce this tendency, but can be incompatible with reinfusion into the patient. An automated blood access system that conserves blood, and maintains patency through the catheter, can operate such that the processes of drawing, measurement, and infusion can be completed within a time frame that prevents damage to the blood and excessive aggregation of blood within the walls of the tubing, sensor and catheter. The time at which these components are allowed to be exposed to drawn blood is referred to as the residence time.

To further maintain patency, remaining traces of blood, left after infusion, can be flushed from the sensor set using maintenance fluids.

Description of Hardware Components

An example blood access system can be described in the context of three component groups, (1) a console, (2) a disposable cassette and circuit and (3) fluid bags that attach to the circuit. Additional information regarding system components is described below (see associated figures for additional information).

Console

Pumps. Pumps provide the ability to move blood and maintenance fluids (typically normal saline) between the patient and the optical sensor. The pumps can be, for example, standard peristaltic pumps which enable bi-directional flow and support stop flow conditions. The example system has two pumps, denoted the blood pump and the flush pump.

Control System. Algorithms that control the pump speeds and directions and measure the sensor set pressures to achieve a complete normal blood access cycle. The normal cycle 1) maintains patency between blood samples 2) withdraws a blood sample 3) returns the blood sample and 4) cleans the sensor set. The control system also provides fluid motion for priming the sensor set as well as detecting fault events in the blood access cycle and can exercise automated procedures to clear the faults, or where not possible, alert the user.

Disposable Cassette and Circuit

Circuit Tubing. The circuit tubing provides for the fluid pathways, including the pump loop tubing and conduits that convey blood and maintenance fluid between the patient and optical sensor. Additional tubing provides for a flush line which provides an effective mechanism for cleaning. The circuit tubing also includes a number of one-way valves that prevent infusion of contaminated saline.

Pressure Sensors. Two pressure sensors measure pressures inside the sensor set near the pump loops. There is a blood line pressure sensor and a flush line pressure sensor. Each sensor measures pressures on the patient side of the pump loops.

Cuvette. A cuvette provides an optical window through which an optical glucose sensor operates.

Blood Reservoir. The dilution of blood with saline as it traverses over the long distance of the sample line requires that the actual draw volume exceed the geometric volume of the blood line to make sure that an undiluted sample is presented at the cuvette at the end of the draw. Additional tubing is provided upstream of the cuvette to provide the additional capacity for blood storage, and this tubing can be wound on a form to provide compactness. This coiled tubing is called the blood reservoir.

Bubble detectors. The blood access system can have one or more bubble detectors that sense the presence of bubbles in various locations in the sensor set. A bubble detector can be present at the sensor set T to detect and prevent bubbles from entering the catheter connection line.

Fluid Bags

Source, maintenance fluid. The blood access system can move blood by virtue of an incompressible maintenance fluid, such as saline, present in the conduits of the sensor set. The sensor set accordingly has a connection to a supply of sterile maintenance fluid, such as a bag of saline. The sensor set is configured such that either the blood or flush lines can draw fluid from the maintenance fluid source, but do not allow fluid, once it has entered the set, to return to the maintenance fluid source.

Waste reservoir. The blood access system can provide a means to dispose of maintenance fluids that are used for blood draws and cleaning. A waste reservoir is provided that accepts maintenance fluid and/or trace amounts of blood cleaned from the sensor set. The waste reservoir is connected such that either the blood pump or flush pump can dispose of fluid, but such that fluid, once delivered to waste, cannot reverse into the sensor set.

A sensor set block diagram for the example blood access system as described above is shown in FIG. 1. The upper right part of the diagram shows a plumbing network that connects to the saline and waste bag. This network contains check valves configured to allow saline to be drawn from the saline bag into either the blood or flush line, and waste fluids to be pumped from either of the same lines into the waste bags. The valves prevent the system from drawing fluid from the waste bag or pumping fluid into the saline bag. From the blood pump fluid can communicate in either direction. On infusion saline is pulled from the saline bag. On withdrawal, fluid from the blood line is pumped towards the waste bag.

Automated Blood Access Process

The example blood access system of FIG. 1 can be operated as described below. Fluid motion is actuated by the pumps in a series of distinct stages that accomplish the functionality described above.

Draw Initialization Stage; Clearing Catheter Access

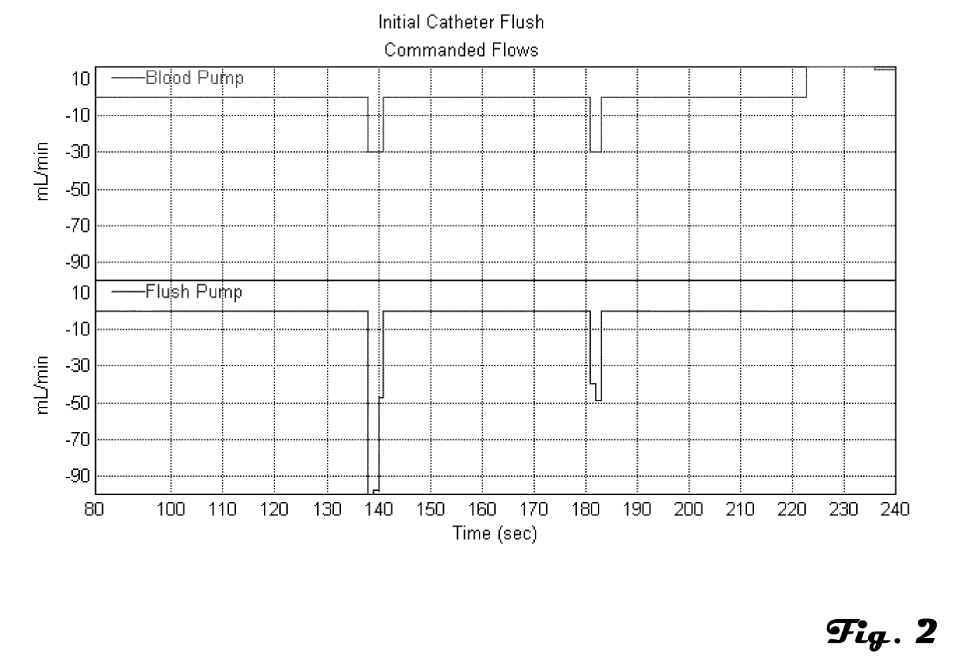

Before the blood draw is started, both the blood and flush pumps are controlled to issue one or more flow pulses at an elevated or maximum flow rate. More than one pulse can be used to insure that at least one pulse will be effective since there is a possibility that the roller may not be fully engaged in the pump race at the start of the pulse. The pulses can clean away any aggregated blood or protein that might have adhered at the catheter tip and any protein sheathing that may have formed during the keep vein open (KVO) stage. To achieve the highest flow rate in the catheter connection line, the pulses in the blood and flush pumps are synchronized. Following each pulse, a delay of about 4 seconds can be used to allow settling of the flow and clearing of the saline that was infused into the vein. FIG. 2 is a schematic illustration of example pulse commands for the blood and flush lines.

Efficient Blood Withdrawal Stage

The blood pump can be used alone to complete the entire blood draw and measurement. To minimize the total draw time about 80% of the total required blood volume is first drawn at a rapid, variable flow rate. A constant pressure-based draw method can be used to compensate for a varying mix of saline and blood, and to achieve maximum flow rate constrained by a constant upstream pressure that keeps fluid degassing at an acceptable level. As blood replaces saline in the blood line, viscosity and therefore resistance to flow increases so that, for a constant upstream pressure, flow decelerates over time. The termination of this stage of the draw can be determined by calculating the % blood dilution or rate of dilution by use of the optical sensor. An optical sensor can sense both the arrival of blood and the component changes as an undiluted sample becomes present in the optical cell.

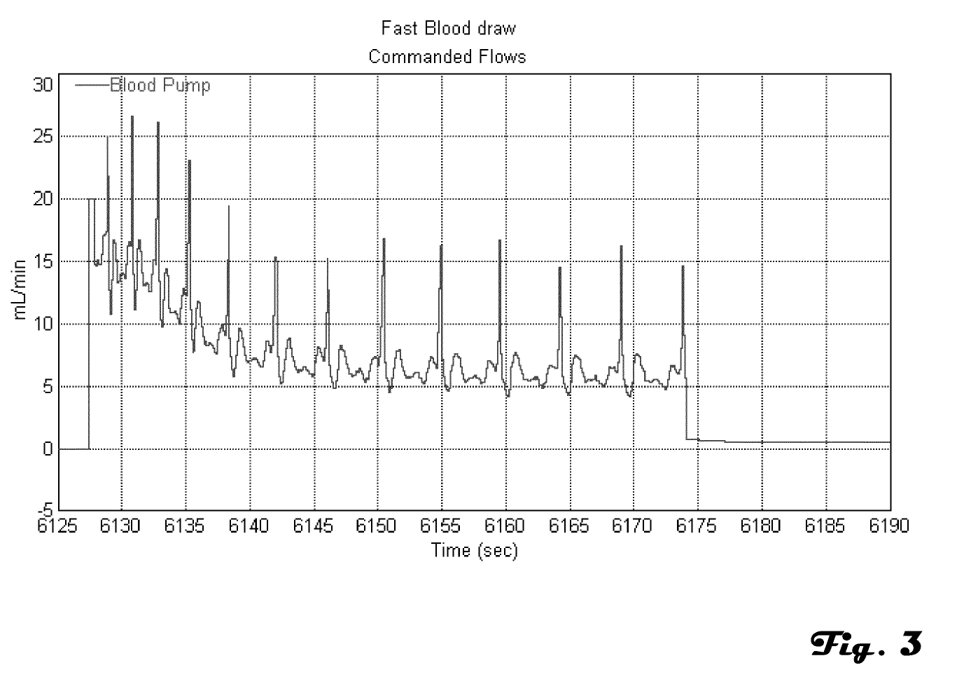

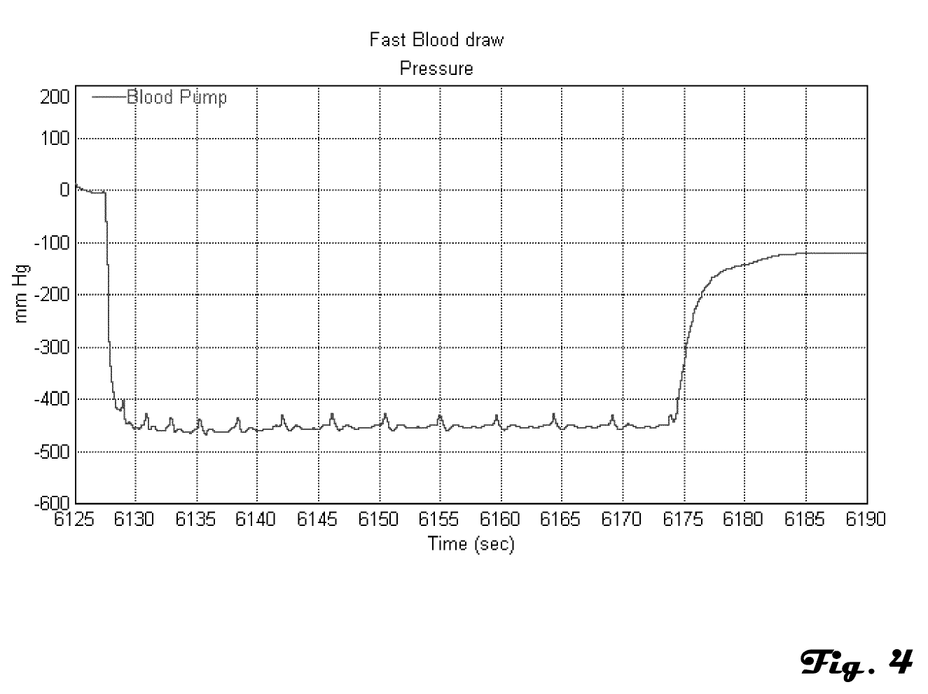

FIGS. 3 and 4 illustrate the flow and pressure, respectively, of a typical rapid blood withdrawal. The spikes on the flow are due to rapid acceleration of the pump through the pump roller events (control dead zone) where the rollers temporarily disengage and cause the pressure to rise (i.e., become less negative). The pressure based servo automatically turns more rapidly to drive this pressure towards the target value, which is −450 mm Hg.

Optical Measurement Stage

Following the rapid draw, the pump flow rate can be slowed to a constant flow rate of about 0.5 mL/min to maintain suspension of the red blood cells in plasma during optical measurement, and to allow sufficient time to complete the glucose measurement. If the measurement period is about 90 seconds, an additional 750 mL is withdrawn.

Infusion Stage

After the measurement is completed, infusion begins as a progression of stages that return the blood as quickly as possible to the patient and also begin the process of cleaning the tubing and cuvette. The initial stage of infusion can use pressure-based control which operates at a variable flow rate to maintain upstream pressure at a high constant pressure. This initial stage infuses almost all the blood that was drawn, leaving a remaining saline-blood mixture at the end of the blood line. This rapid infuse stage limits the total time that blood is in the extracorporeal circuit. As an example, the first stage of the infusion can be completed within three minutes.

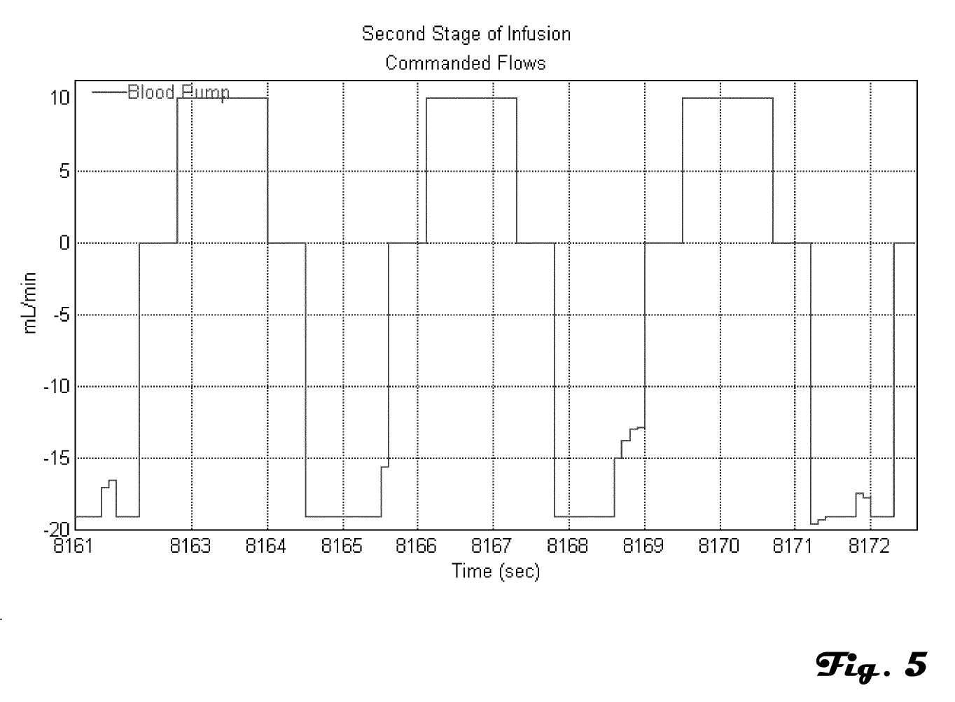

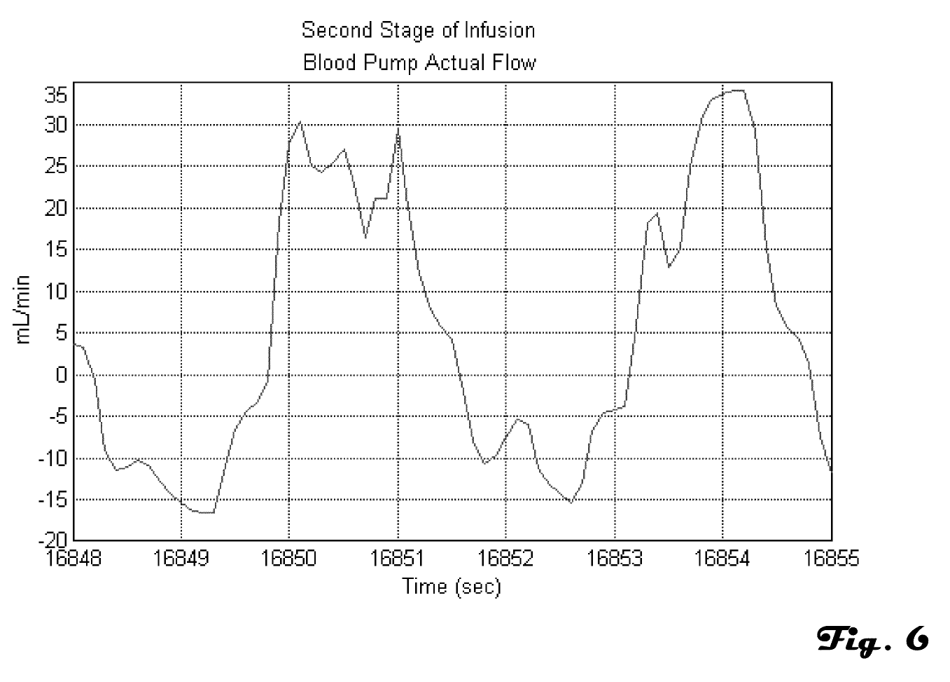

The next stage of infusion involves a repetitive back and forth motion of the blood pump such that during half of one cycle, the pump pushes blood forward at a constant flow rate and during the second half of the cycle blood is pulled back at about half the rate. Between each forward and backward movement, the pump is briefly held motionless to allow flow to decay to zero. The asymmetric cycle results in a net forward movement of fluid. Since the tubing diameters do not under any operating circumstances permit operation in a turbulent flow regime, the acceleration and deceleration of fluid results in eddies that disrupt the fluid boundary layer which help wash away any cells or other blood products that could potentially adhere to the tubing walls. During this stage of infusion, and if pressure exceeds maximum limits in either direction, the flow is throttled back to limit the pressure. An example commanded flow for this stage of infusion is shown in FIG. 5. Here the negative flow (towards the patient) is partially limited by pressure. FIG. 6 shows the actual flow at the catheter during this stage of infusion. The difference in the commanded flow profile and the actual measured profile is the compliance of the circuit. The major source of compliance in the circuit is the pump loops.

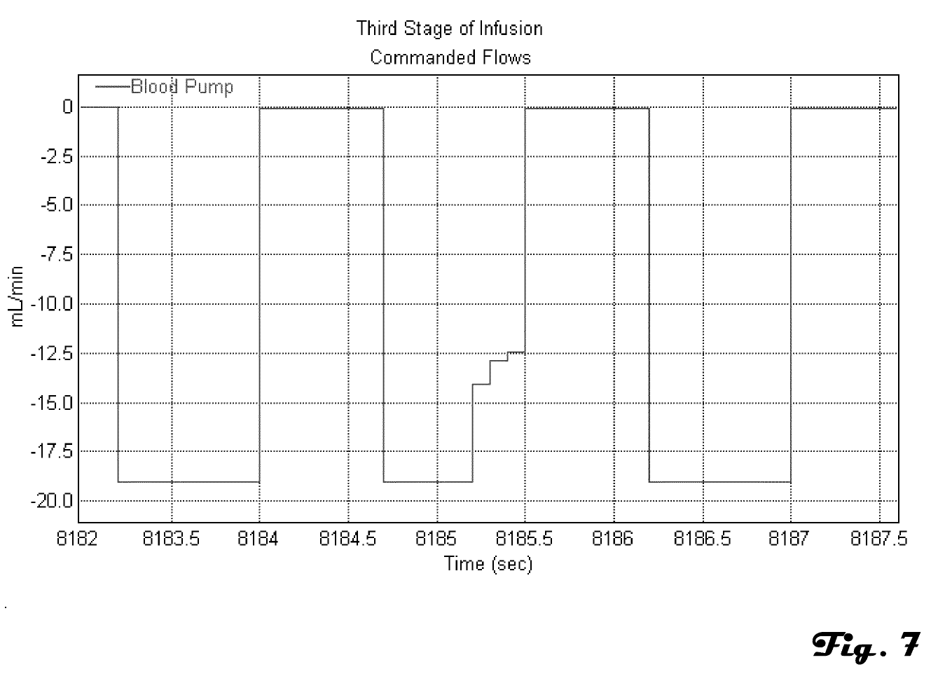

Following the 2^{nd} stage of infusion, a 3^{rd} stage begins with the blood pump executing a repetitive forward-pause motion with 50% duty. Although this stage might not be as effective as the forward-reverse flow, it still provides some pulsatile acceleration and washing of blood products from the tubing walls, while providing a higher net forward flow rate per cycle. The flow in this stage is also limited by pressure. FIG. 7 illustrates the third stage of infusion commanded flow.

Cleaning Stages

At this point in the cycle, in the example embodiment greater than 97% of the blood has been returned to the patient; the next stages focuses on a more thorough cleaning of the cuvette, tubing and catheter.

‘Scrub’ Stage

The scrub stage involves rapid, reverse-synchronized back and forth motion of the blood and flush pumps so that fluid movement commutes only between the blood and flush lines. Total volume moved per cycle is only about 300 uL and the flow is not turbulent, but the rapid oscillations can create accelerations that lead to eddies that circulate in a cross flow direction and help to wash blood products that collect in the boundary layer and on the walls of the tubing and cuvette to mix with the central stream.

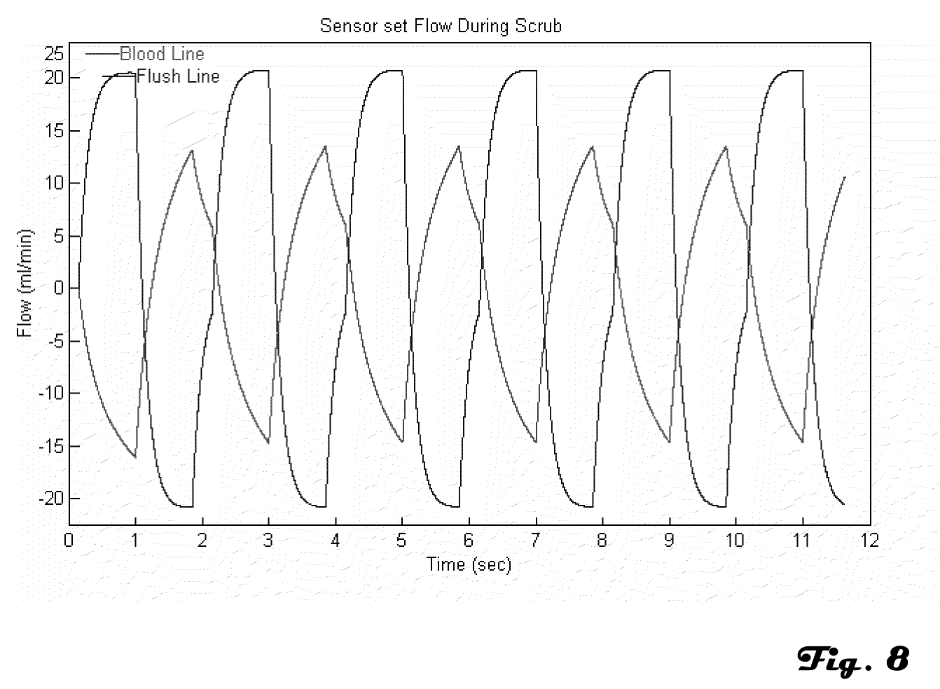

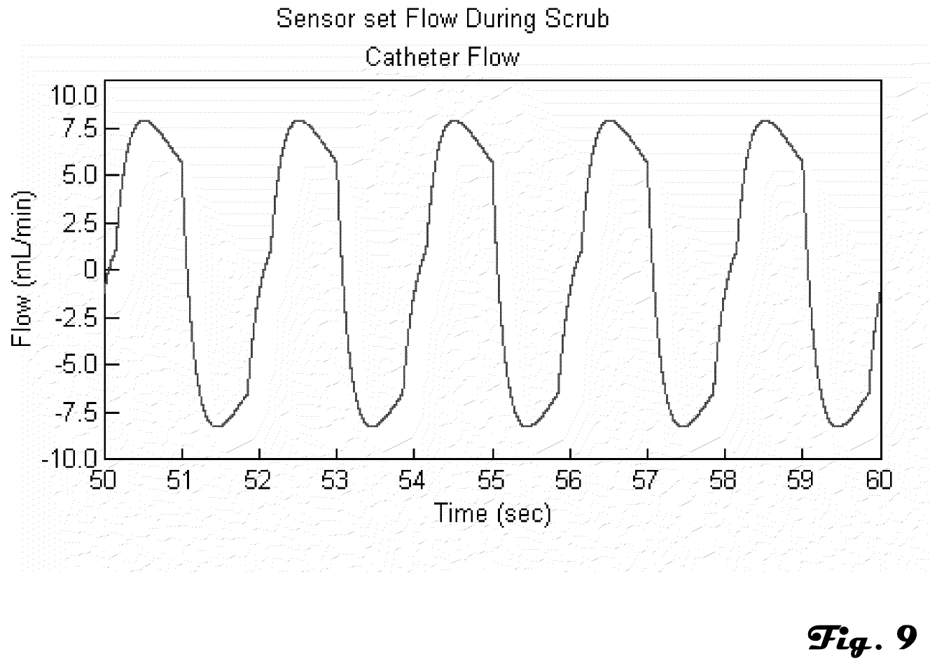

The blood line inside diameter can be sized to minimize the draw time for blood. This diameter optimization takes into account flow resistance, volume, flow velocity, and shear. The flush line on the other hand can be sized at a much larger diameter to provide a conduit for executing high flow rate maneuvers to clean the catheter tip. The relative size difference does not necessarily interfere with any fluid motion functions that require infusion and withdrawal. However, during the Scrub stage, which requires circulation between the lines, and where a zero flow is desired at the catheter, the relative size between the blood and flush lines can create difficulties, especially during transients. Although the blood and flush pumps have identical flow commands, the higher resistance of the blood line slows the actual flow response compared to the flow response in the flush line. FIG. 8 illustrates the flow differences present during the scrub stage of cleaning. FIG. 9 illustrates actual fluid flows in the catheter. The flow imbalance is undesirable since the net flow can pull blood back into the sensor set during cleaning necessitating repeat cleaning.

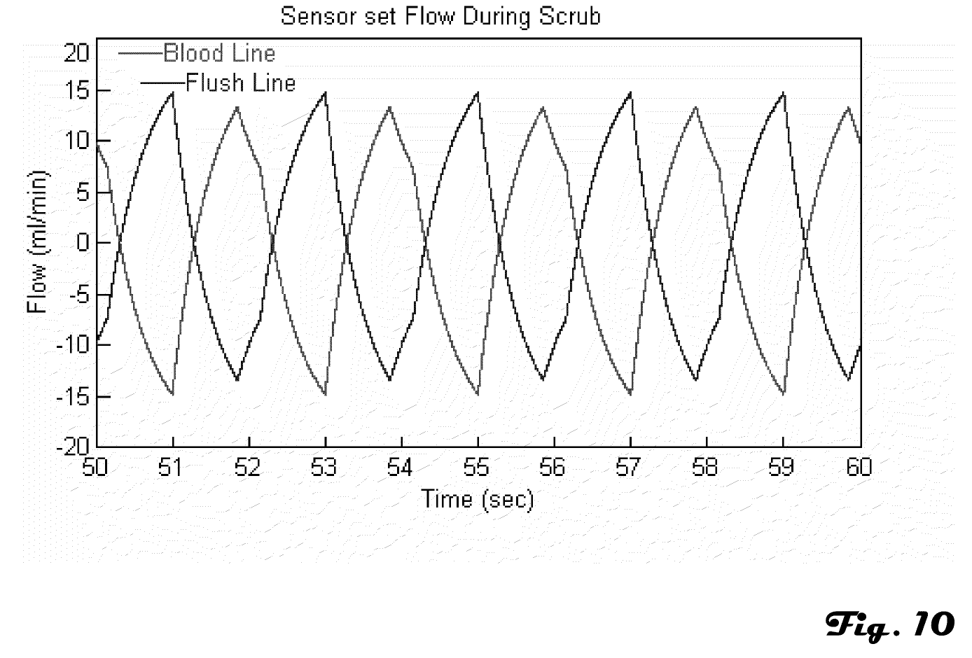

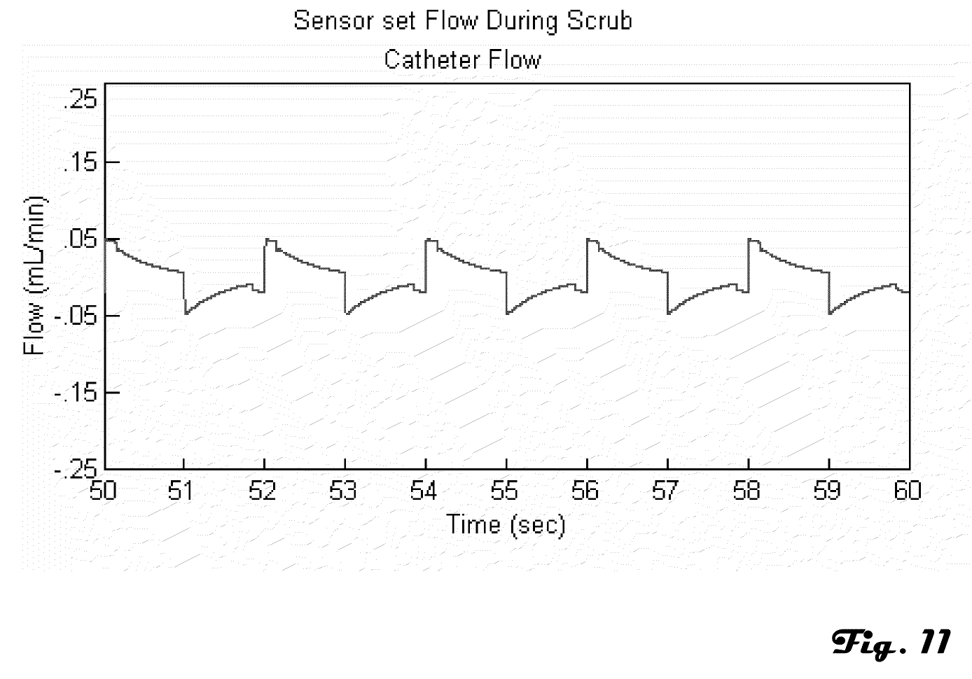

Balancing flow rates in the blood and flush lines can be accomplished by estimating the pressure at the sensor set T junction and using the estimated pressure in feedback to control zero pressure or the expected pressure head at the catheter tip. The process of dynamic balancing results in almost zero flow through the catheter connection line. The result of this control method is illustrated by FIGS. 10 and 11 which show the balanced blood and flush line flows during balanced control.

Recirculation Stage

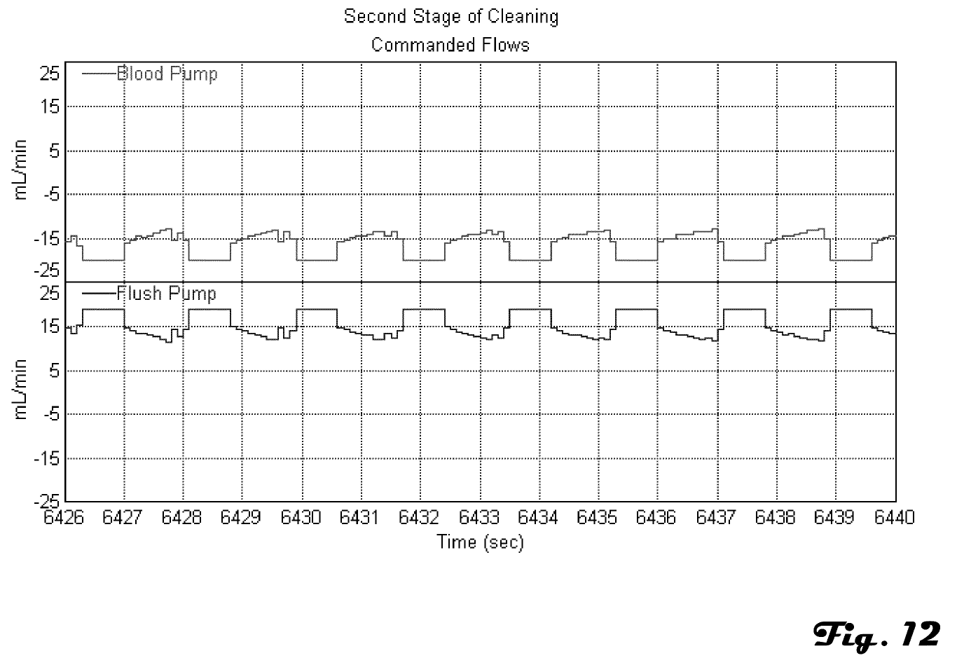

Once blood products have been washed into the mainstream by the scrub stage, the blood and flush pumps can be operated at a constant rate such that the bulk of fluid movement occurs from the blood line to the flush line. This washes the remaining blood products in the blood line and flush line towards waste, and filling both legs of the set with clean saline. The blood pump is operated at a slightly higher rate so that a small amount of fluid is driven out through the catheter connection line to hold blood back and to begin cleaning of the extension line and the catheter. FIG. 12 illustrates the blood and flush pump commands during an example recirculation stage of cleaning.

Catheter Flush Stage

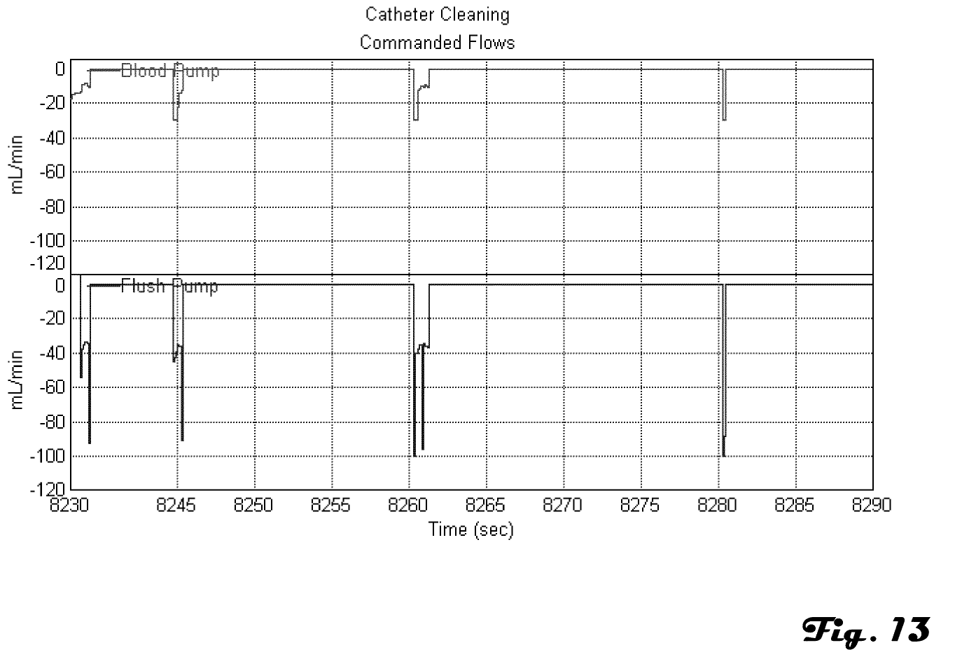

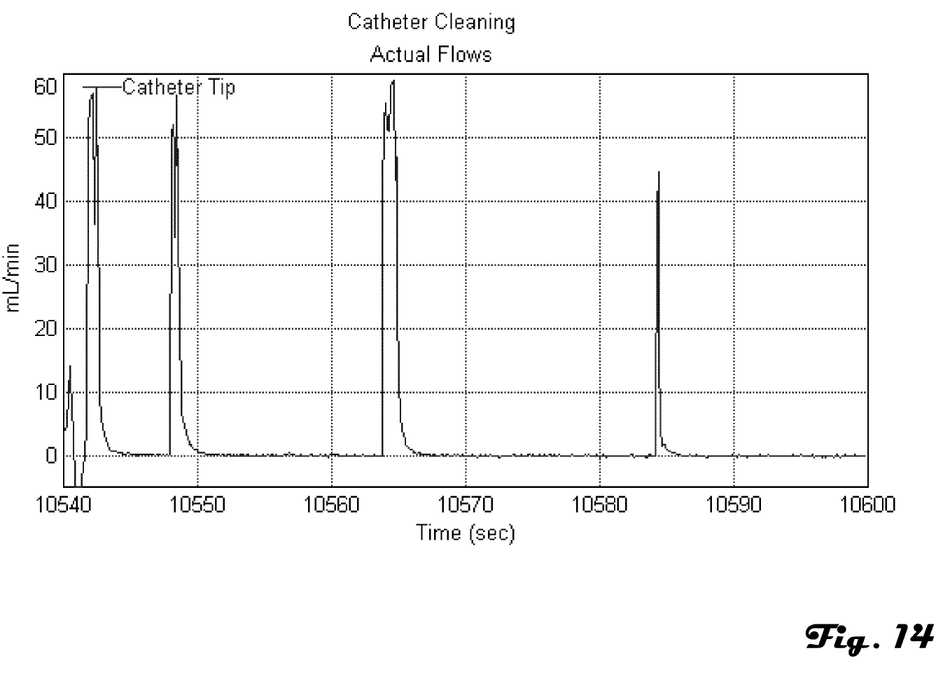

The final stage of cleaning the sensor set involves high flow pulses with larger volume pushes to completely clear the catheter connection line, Luer connections and catheter. The blood and flush pumps can be used in synchronization with each other to obtain an effective cleaning flow rate. Delays can be used between the pulses to allow settling of the flow before the next pulse is issued. Since the flush line has the larger inner diameter, the highest flow comes from that line. FIG. 13 illustrates the flow commands to the blood and flush pumps during the catheter flush and FIG. 14 shows the actual flow from the catheter tip.

Keep Vein Open (KVO) Stage Between Measurements

The system can operate in a KVO stage during the period between measurement cycles. The KVO stage provides a constant flow rate into the patient at a rate that prevents blood from entering the catheter, maintaining an open blood access connection between blood draws.

Frequency of Measurement Cycles

The highest frequency at which measurements can be obtained is limited by the ability to complete the normal sequence of withdraw, measurement, infusion and cleaning stages and a minimal acceptable KVO time. An example system has been developed and tested that supports a measurement frequency of one measurement every 7 minutes.

Exceptional Detection and Management

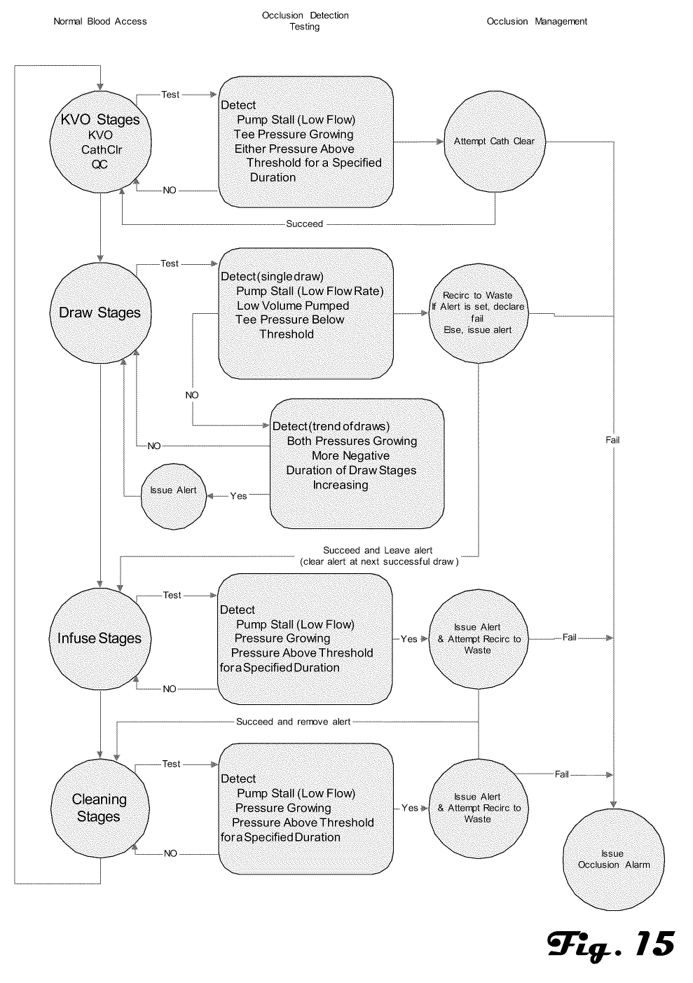

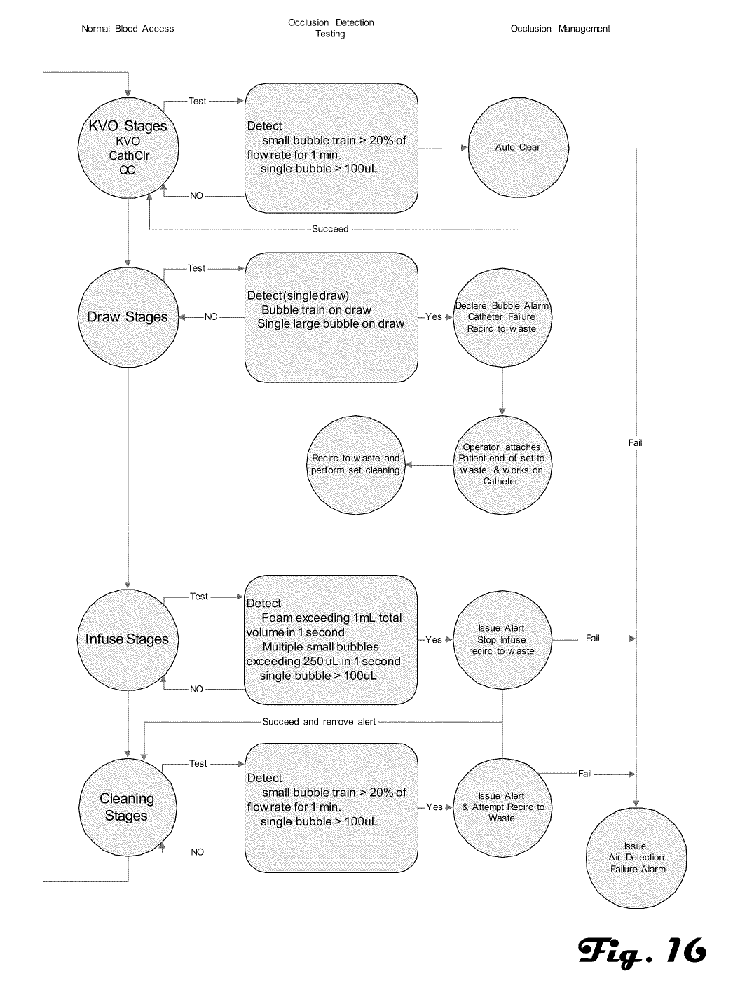

Occlusions and air bubbles can occur during the operation of a blood access system. Embodiments of blood access systems in accord with the present invention can detect and manage occlusions, restrictions, and air bubbles that occur during operation of the system. These operational problems can occur during any operational phase. As the recovery or management of a given exception is dependent upon the stage or operation, the system can provide different recovery methods depending upon the stage of operation. By the use of two pressure measurements, the system has the ability to identify the location of the problem so that effective procedures or alarms can be provided and allow effective resolution of the problem. FIGS. 15 and 16 comprise decision flow charts that can be used for occlusion detection and management as well as air bubble detection and management.

Patency Evaluation.

Embodiments of the present invention can determine the patency of a blood access site using determinations of the actual pressure and flow characteristics of a particular patient and comparing this to an envelop of all pressure flow characteristics determined for an IV configuration and pumping profile. Embodiments of the present invention can determine the patency of a blood access site using comparisons of the actual pressure and flow characteristics of a particular draw of a patient against the historical data of pressure and flow from this patient.

An example embodiment of the present invention uses 8 different techniques to determine the patency of blood access site. A similar 8 techniques can be used for occlusion detection, however the thresholds for occlusion detection can be different than the thresholds of patency evaluation.

There are two elements in the evaluation of the patency of a blood access site with a blood access system operated under flow control:

Flow One: The instantaneous pressure required to push or pull the fluid through the access site at known rates.
Flow Two: The draw to draw evolution of the pressure required to withdraw or infuse fluid into the patient.

There are two elements in the prediction of the patency of a blood access site operated under pressure control:

Pressure One: The minimum sustained flow rate achieved at the desired pressure.
Pressure Two: The evolution of the sustained flow rate from draw to draw.

There are four elements of patency prediction which are common for both pressure and flow based controls:
Decay One: The decay of pressure generated when flow is stopped
Decay Two: The evolution of the decay of pressure when flow is stopped from one to draw to another on a single patient.
Growth One: The increase of pressure created when flow is started or increased.
Growth Two: The evolution of the increase of pressure created when flow is started or increased from one draw to another on a single patient.

Use of Pressure and Flow Data.

A blood access system can be programmed to withdraw and infuse at known conditions of pressure and flow. These conditions can be tested in vitro to generate maximum and minimum envelopes of pressure and flow for a particular system. When a new patient is attached to the device, the first blood access event can be compared to these envelopes to make a prediction of the patency of the blood access site. The more data known about the individual patient, the more accurate the patency prediction becomes. In particular, knowledge of the hematacrit of a patient can greatly improve the specificity of the prediction.

The relationship between flow and pressure in many blood access systems can be expressed in a linear equation know as Poiseuille's Equation:

Flow(Q)∝Pressure*(radius of tube)**4/(viscosity*length of tube)

Once the design of a blood access system is complete, the geometric terms in the above relationship are fixed and pressure/flow relationship becomes inversely related to viscosity:

Flow(Q)∝Pressure/viscosity

This simple relationship is at the core of all existing occlusion management systems. To understand the patency evaluation of an access site, the relationship should be rewritten as:

Viscosity∝Pressure/Flow(Q)

The viscosity of a given patient's blood is normally constant and strongly dependent up on hematocrit. An individual patient's blood viscosity can be estimated using the pressure and flow data generated in the blood access system. From draw to draw on a given patient, the estimated viscosity should remain substantially constant and should be in a normal range for humans. Increases in the apparent viscosity are signs of degrading patency. The example system uses algorithms which evaluate the draw to draw evolution of pressure and flow to determine when patency is degrading.

The system can respond to these changes by performing extra cleaning cycles and extra catheter cleaning pulses to re-establish the initial level of patency. If patency cannot be increased by these methods the system alerts the medical personnel of impending failure so they can try to restore blood access prior to actual failure.

The relationships between pressure and flow hold well over all of the normal operating regions for blood access systems, however, the more complicated the system, or the closer to occlusion the system becomes, the worse the estimate of performance using Poiseuille's Equation.

Robustness of Patency Prediction.

The example patency prediction method relies upon two variables which are closely related due to the hemodynamics of the blood access system: the flow and the pressure. A reasonable prediction of patency or impending occlusion benefits from a minimum of two “independent” variables for implementation. An additional element which can make the prediction more robust is the use of trend data generated from a specific patient.

Pumping elements and other non-tubular sections of a blood access system do not always follow Poiseuille's Equation. In vitro test data can be used to hone the limits of the prediction and build robustness into the predictions. Some embodiments of the present invention have elements which can increase the accuracy and specificity of the patency evaluation. In particular, dual pressure sensors connected close to the access site provide a highly accurate measurement of the pressure at the catheter when one of the pumps is stopped. Also optical sensing elements allow a reasonable estimate of hematocrit which allows the prediction to be further enhanced.

Implementation.

Any blood access system which generates known flow rates and contains at least one pressure sensor can implement patency prediction according to the description herein. The accuracy of the thresholds, and the specificity of the prediction can depend on the specific configuration and implementation of the blood access system.

Example Implementation.

Using the example blood access system of FIG. 1, and pumping profiles like those described herein, some important properties of a patent blood access site are:

Appropriate blood flow capacity to support the rate of fluid movement.

3.5 mL withdrawn in 30 to 60 seconds

Peak withdraw rates of 15 mL/min

Peak infuse rates of 15 mL/min

Structurally sound to handle associated pumping pressures

Withdraw at down to at −300 mmHg

Infuse at up to +300 mmHg

Not Position sensitive

Catheter not near wall

Catheter not near valve

Site is not Infiltrated

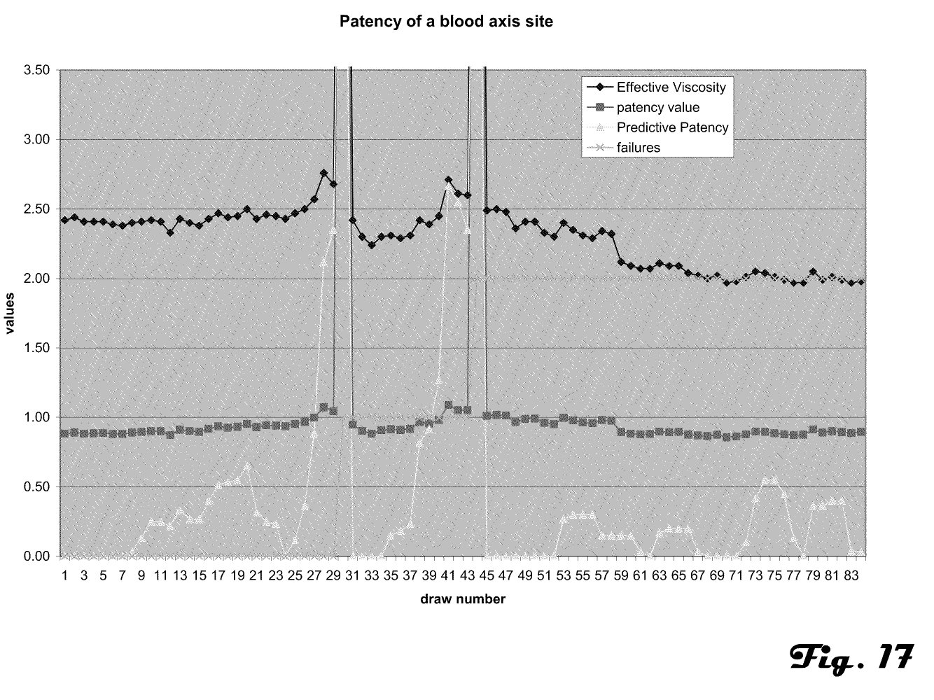

FIG. 17 shows the method implemented on blood draws performed on a swine. The effective viscosity is determined from the average rate of withdraw of the blood. The patency value is shown as the effective viscosity divided by hematacrit. This value has been scaled so that a patency value is above 1.0 indicates that the patency of the access site should be checked. Also plotted is the patency prediction. This value has similarly been scaled so that values above 1.0 predict future failure of the blood access site. The final line is a counter showing the total number of failures the particular blood access site experienced. While this data was gathered, the predictive element and the preventative cleaning were disabled so the accuracy of the predictions could be tested. Both of the indicated failures required extensive manual intervention to reopen the access site. These methods predict the failure of a blood access site from 3 to 7 draws before the failure occurs. Because most blood access systems are used infrequently, once and hour or every 30 minutes, this prediction gives medical personnel adequate time to clean the blood axis site if the automated cleaning methods do not prevent degradation.

Occlusion Prediction.

A blood conserving blood access system generally requires staged flow operations that 1) draw and transport the blood sample for analysis and 2) infuse the blood back into the patient and 3) clean remaining blood residuals from the system. Aggregation of blood components on the walls of the fluid transporting conduits or clots which can lodge in conduit joints and partially occlude flow can eventually lead to full occlusions of the conduit and failure of the blood access. If the system is able to measure properties within the conduit affected by occlusion, it can predict the onset of these full occluding events, and furthermore the specific location where these events are occurring. This information can be used to warn the caretaker, who subsequently can initiate mitigating actions to restore full patency and avoid failure of the blood access. Alternatively, the system itself can directly use this information and perform automatic maneuvers to mitigate the issue.

An example embodiment of the present invention including an ability to predict the onset of an occlusion assumes the two pump architecture of FIG. 1 with two lines that connect together with the catheter connection near the patient and where each pump has its own independent pressure sensor. One of these pump lines (the blood line) performs the function of draw and infusion of blood and the fluid in the other line (the flush line) remains static. Since the fluid in the flush line remains static, the flush pump pressure sensor is able to accurately and remotely measure the pressure at the catheter connection line's junction with the blood line. The blood pump pressure transducer measures the draw and infusion pressure of the blood pump.

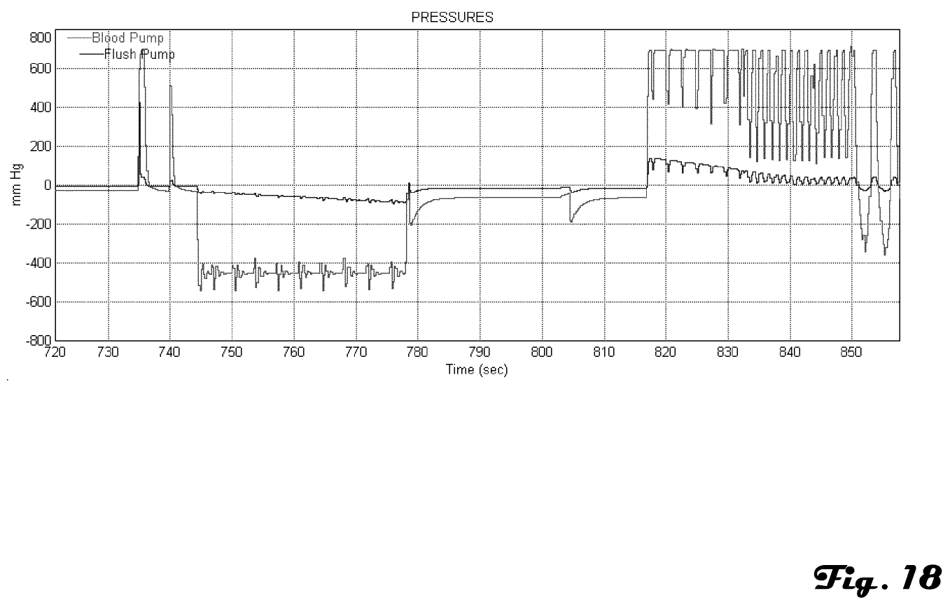

FIG. 18 illustrates these two pressure measurements during a normal draw and infusion cycle. The red trace represents pressure at the blood pump, and the blue trace is the flush pump pressure which measures the pressure at the catheter connection since the flush pump is not moving. The draw begins at about 745 seconds and completes at about 778 seconds. The infusion begins at about 817 seconds and ends at about 832 seconds. The time between draw and infusion continues to draw blood but at a decreased flow rate for to complete a measurement. After 832 seconds the system further infuses blood, and begins to clean the blood line. In this particular instance, the draw and infusion stages are pressure targeted in that the blood pump is controlled to regulate at constant negative and positive pressures, respectively. Note that during the draw stage, for a non-occluded catheter, the flush pressure responds with a minor negative deflection, and similarly during the infusion stage there is a positive deflection of the flush pump pressure. As long as the catheter connection remains constant, the pressure signature in each of these stages will repeat for each subsequent cycle.

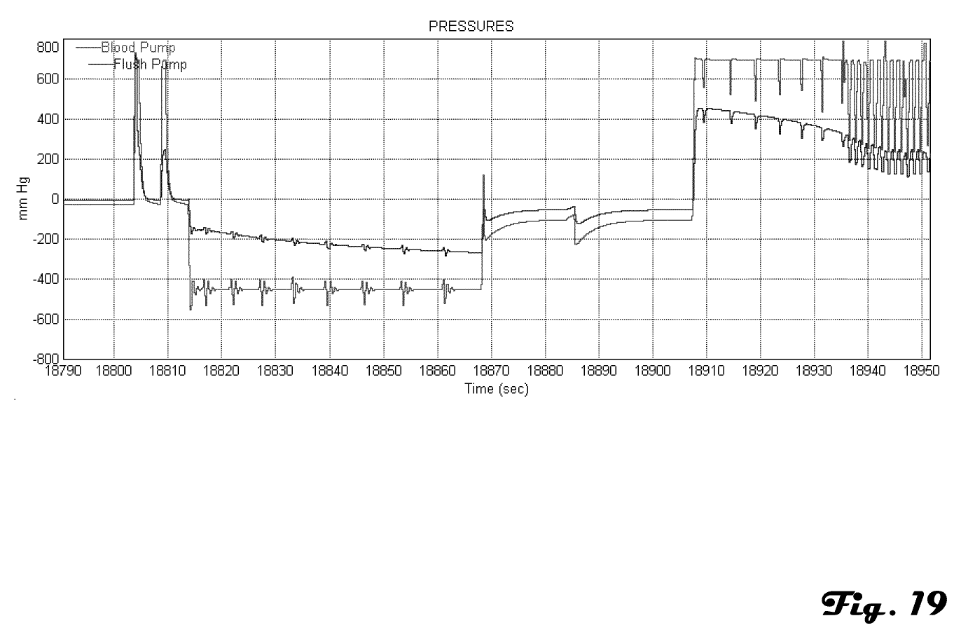

In the event blood components begin to deposit in the catheter connection line, the signature of the flush pump pressure begins to change with each successive cycle until the flow resistance becomes significant to the point it's considered a partial occlusion. FIG. 19 illustrates the state of the pressures at an advanced time where blood has aggregated in the catheter. Additionally the pump rotational rates have slowed.

These two figures illustrate, and motivate the means for predicting the onset of either full or partial occlusion. The method first records an initial pressure deflection metric from the draw and infusion stages. Then for each cycle, the metric updates a filter that estimates the rate of change of flush pressure for the draw and infusion stages. By testing this rate of change against a threshold rate, the system is able to decide and alert the caretaker of an impending occlusion, and furthermore if the occlusion is specific to draw or infusion. Similarly, the initial blood pump pump-rate and the rate for each cycle during draw and infusion is recorded. The rate of change of the pump rate can then be determined from each cycle to another and evaluated against a threshold and used to corroborate the pressure information.

An example embodiment of the present invention can provide a similar implementation of the method described above using draw-infuse cycles that, rather than using pressure targeted draw and infuse stages, use constant flow targets. In this example, the blood pump pressure is not constant as occlusion resistance increases. In this example, since the blood pump flow is the independent variable, it is not used to predict, however the blood pump pressure rate of change (on a cycle by cycle basis) is the predicting variable. Furthermore, if the occlusion proceeds between the blood pump and the junction of the flush line, the expected behavior of the blood and flush pump pressures differ such that the onset of occlusion is predicted as well as its location (in the blood line rather than the catheter connection).

The expected trends depending on the type of blood access (pressure or flow based) and location are summarized below:

| Occlusion Location | Pressure Based | Flow Based |
|---|---|---|
| Catheter Connection | Pf →Pb | Pf →Pb |
| | Qb decreases | Pb increases |
| Blood Line | Pf normal | Pf normal |
| | Qb decreases | Pb increases |

Predicting Occlusions in Phase Space.

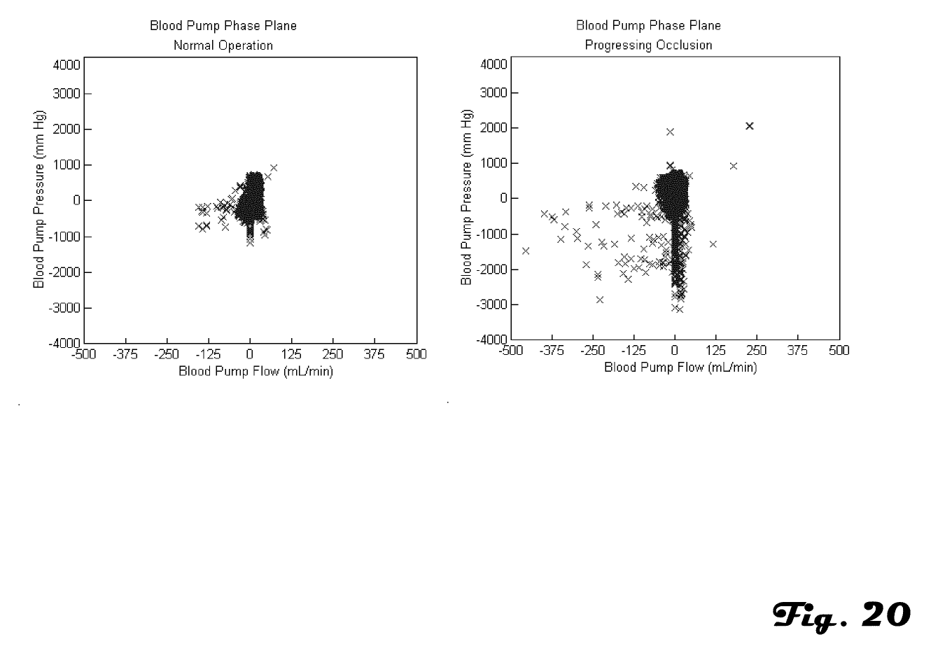

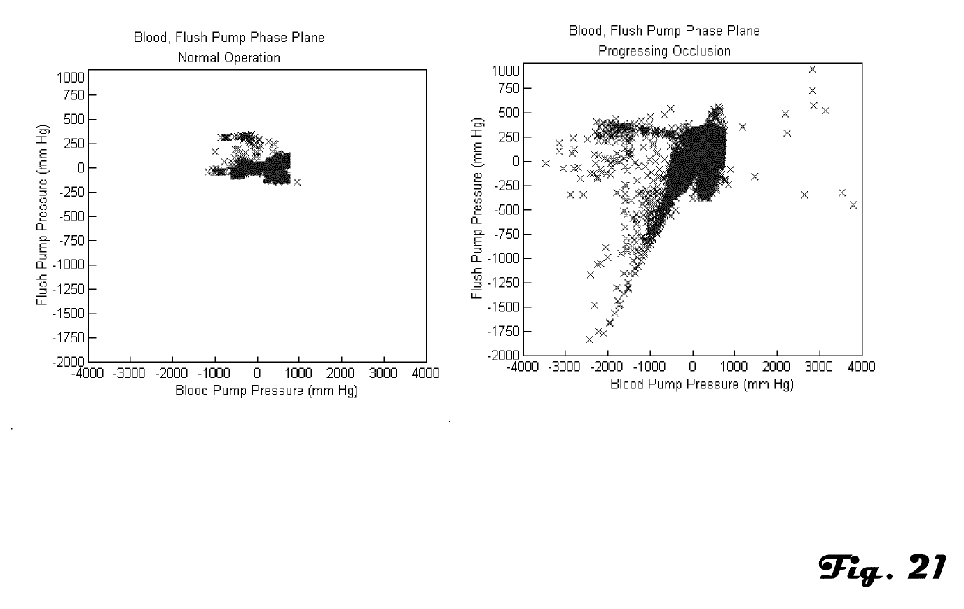

The methods described above depend on time as a variable; however time and rates of change are not necessarily required to predict the onset of occlusion. Pairs of measurements that are compared independent of time are known as phase-plane or phase space analyses. With continuous, normal operation of the blood access controls, the phase-space relationship between variables (eg. Blood Pump Pressure—Flush Pump Pressure or Blood Pump Pressure—Blood Pump Flow) is repeatable on a cyclic basis. As an occlusion begins to manifest itself within the flow path, this phase plane signature begins to deviate, and by using an appropriate phase—plane envelope as a threshold, can be triggered to alert the operator of an impending occlusion. FIGS. 20 and 21 illustrate examples of the phase space progression comparing flows—pressure and pressure—pressure relations respectively from normal operation to near full occlusion.

Example Blood Access System Embodiments.

The methods, features, and elements described above in the context of some example blood access systems can also be implemented in other blood access systems, with appropriate modifications that those skilled in the art will appreciate based on descriptions herein. The terms “sensor” and “sensor system” as used herein are broad terms, and are to be given their ordinary and customary meaning to a person of ordinary skill in the art (and are not to be limited to a special or customized meaning), and refer without limitation to a device, component, or region of a device by which an analyte can be detected, quantified, or both.

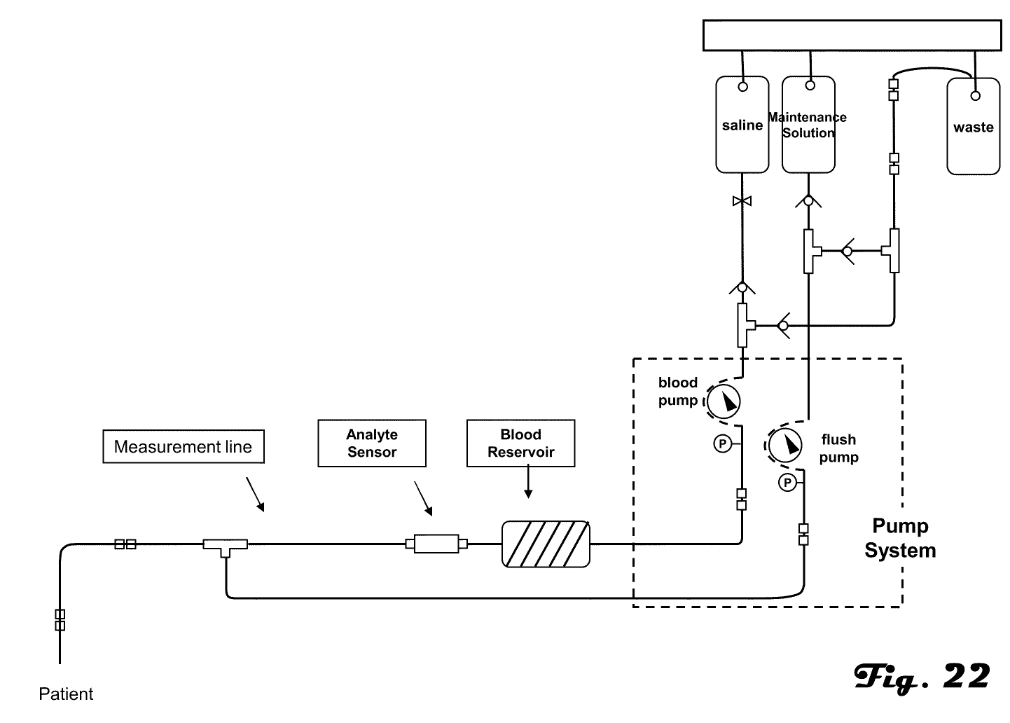

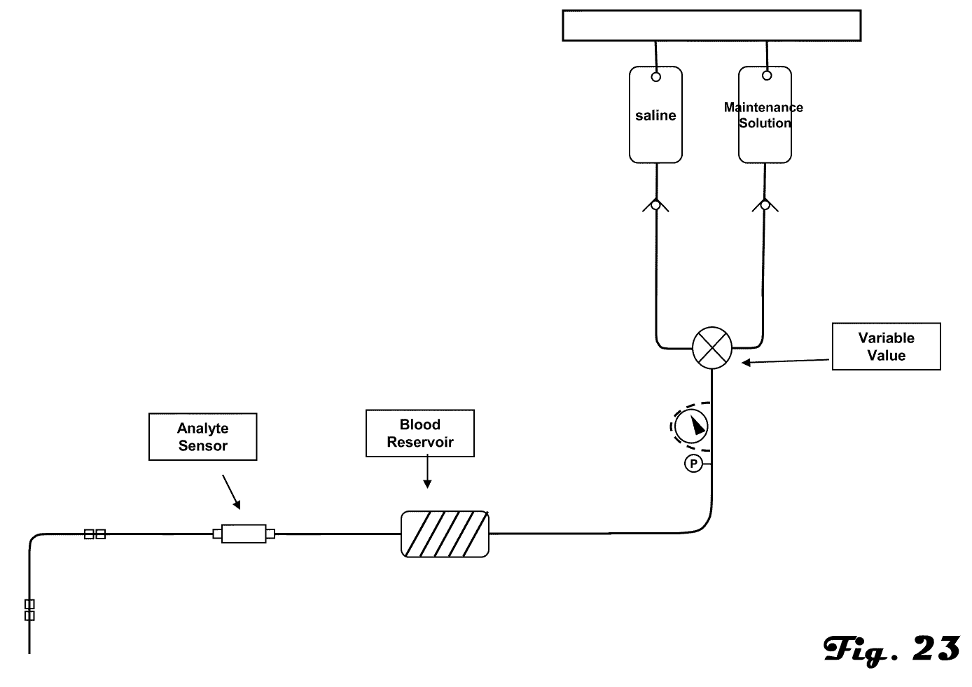

FIG. 22 is a schematic illustration of an automated blood access system that contains two fluid bags providing for at least two different calibration points. In practice the blood is pulled from the patient via the measurement line to the analyte sensor. The analyte sensor makes a measurement of the analyte and a portion of the blood is returned to the patient. In use, the analyte sensor can be exposed to a zero or predetermined glucose concentration via the saline bag. A second glucose concentration can be provided via the maintenance solution bag. The system shown in FIG. 22 provides the opportunity for calibration of the device with a known fluid while concurrently minimizing the infusion of said fluid into the patient. As shown, the maintenance fluid solution can be pumped through the circuit and directly to waste without infusion into the patient. Specifically, the flush pump can be operated in a manner towards the patient and the blood pump can operate at a similar rate away from the patient. In this manner the analyte sensor is exposed to the maintenance fluid solution but no fluid is infused into the patient. Following sensor calibration, fluid for the saline bag can be used to wash the circuit and a similar manner. Such a process enables the effective calibration of the glucose sensor. Such a system also provides the opportunity to clean or maintain circuit performance with additives where infusion into the subject is not desired.

The system shown in FIG. 22 is also well-positioned for use of citrate as the anticoagulant. One example embodiment involves placement of citrate in the saline bag as it is the fluid that makes the most contact with the blood. Contact with citrate effectively anticoagulates the blood during operation of the circuit. If there are concerns regarding binding of calcium at a high level, calcium can be added to the maintenance bag and infused into the patient during those periods between measurement.

FIG. 23 is a schematic illustration of an implementation of a two level sensor calibration device. The system enables the analyte sensor to be exposed to at least two known glucose concentrations. The variable valve can be a simple stopcock where the solution provided to the analyte sensory is 100% maintenance solution or 100% saline solution. In additional embodiments the variable valve can provide for careful mixing of these two fluid solutions so as to create multiple glucose concentrations.

FIG. 23 shows an example implementation of a blood access system with a two level sensor calibration device. This system uses a continuous piece of tubing between the patient and the sensor so as to provide a single fluid path. There is no requirement for a single piece of tubing but the flow path does not have bifurcations. The system enables the analyte sensor to be exposed to at least two known glucose concentrations. The variable valve can be a simple stopcock where the solution provided to the analyte sensory is 100% maintenance solution or 100% saline solution. In other embodiments the variable valve can provide for careful mixing of these two fluid solutions so as to create multiple glucose concentrations.

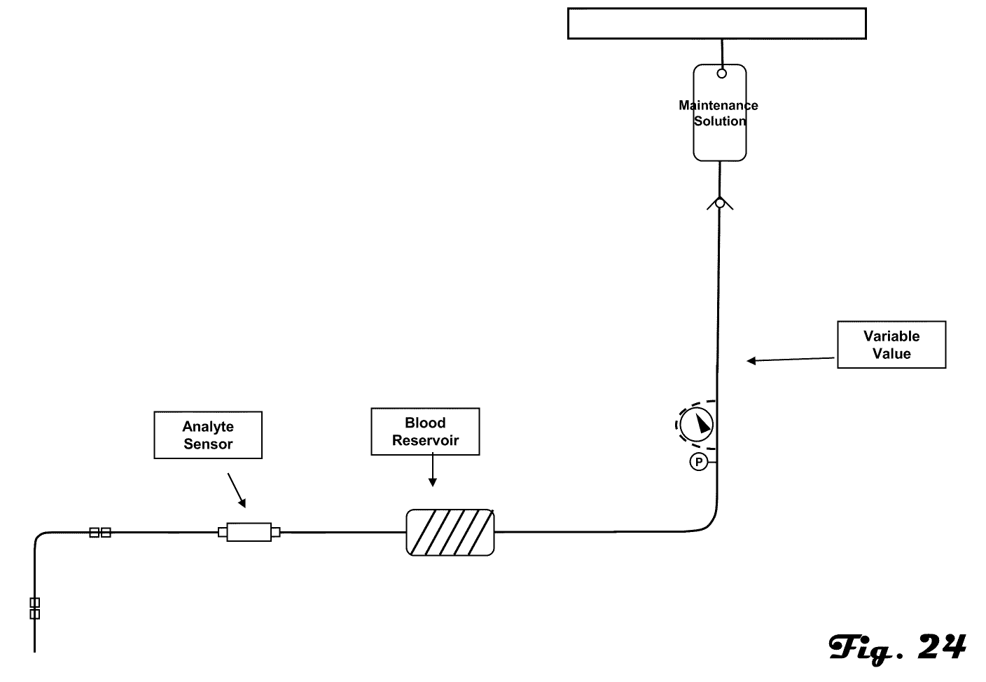

FIG. 24 is a schematic illustration of an example implementation of a blood access system with a single level sensor calibration device. The calibration solution can be zero glucose (saline) or other values of glucose. This system uses a continuous piece of tubing between the patient and the sensor so as to provide a single fluid path. There is no requirement for a single piece of tubing but the flow path does not have bifurcations. The system enables the analyte sensor to be exposed to one known glucose concentrations.

FIG. 25 is a schematic illustration of an example blood access system that enables mixing of glucose into the blood obtained from the patient. This circuit design can enable calibration of the analyte sensor at two known glucose concentrations as defined by the maintenance solution and the saline solution. In addition to providing the glucose sensor with non-blood based calibration solutions this system enables the calibration of the device using blood. In operation in the blood sample can be withdrawn from the patient and exposed to the analyte sensor. Following this baseline measurement a predetermined amount of glucose can be added to the blood is it is pushed back towards the patient. This additive amount enables recalibration of the sensor. The system has the ability to create multiple glucose levels in both saline based calibration standards as well as defined difference blood based calibration standards. The ability to manage the amount of mixing occurring at the T-junction and the corresponding glucose concentration at the analyte sensor is controlled by the variable valve and pump.

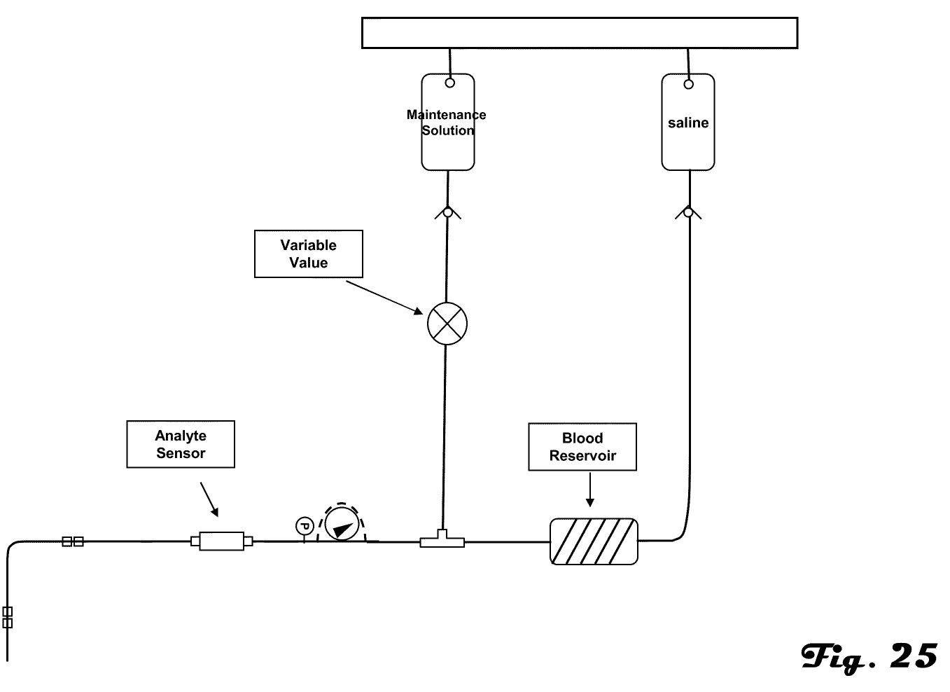

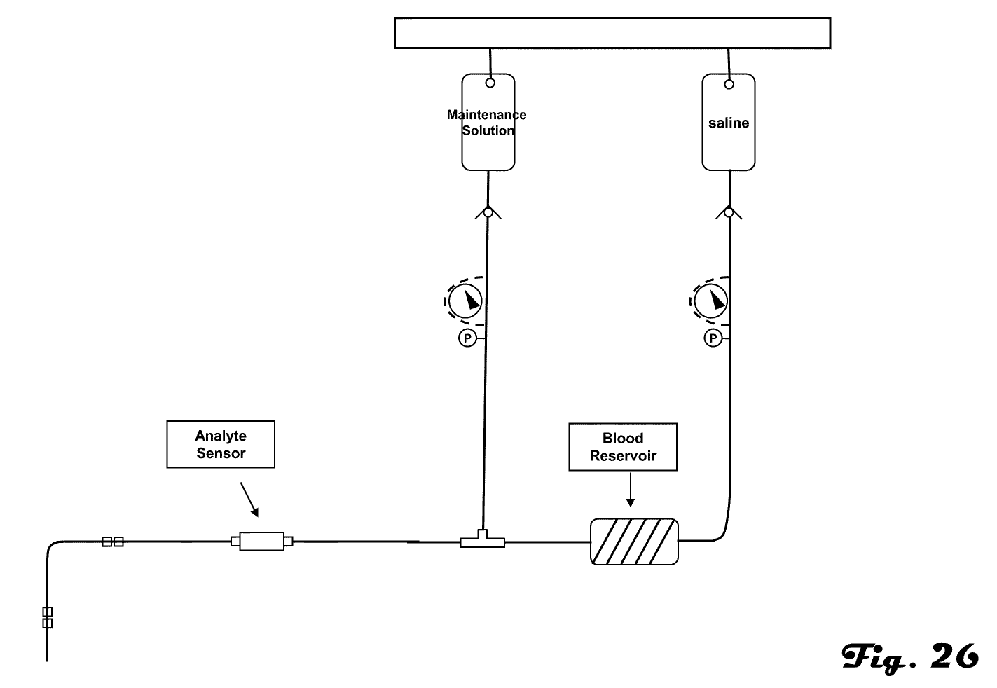

FIG. 26 is a schematic illustration of an example embodiment. The example embodiment contains two pumps. As shown in FIG. 26, these pumps are peristaltic pumps. Peristaltic pumps enable bidirectional flow as well as support stopped flow conditions. The blood access circuit shown has the ability to perform a two point saline based calibration as well as defined glucose additions to the blood sample. The two pumps and reservoir provide the opportunity for assuring good mixing of the glucose throughout the sample.

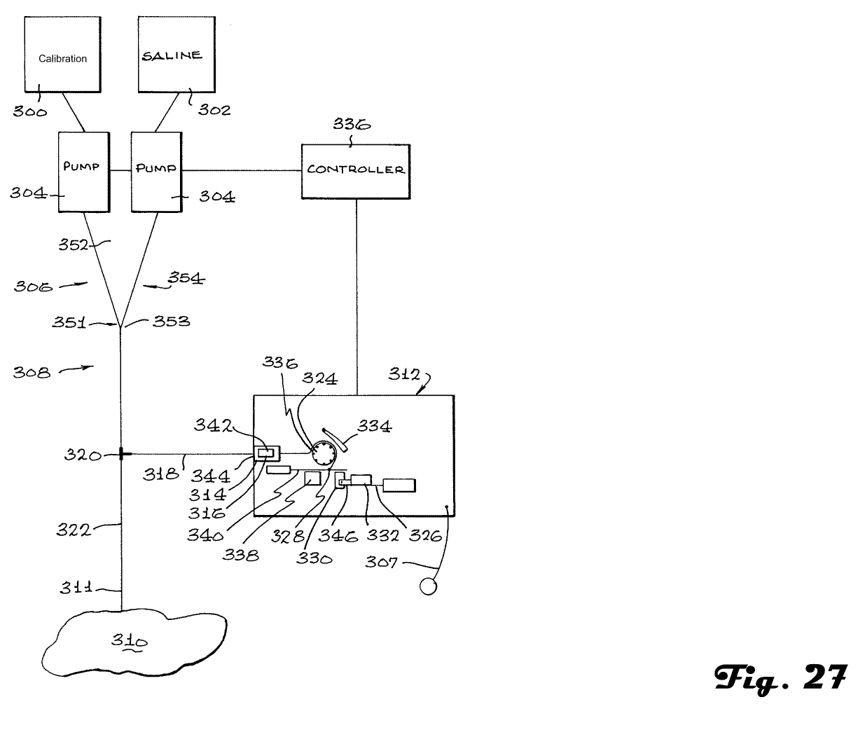

FIG. 27 is a schematic illustration of an example blood access system embodiment. In this embodiment, a calibration solution 300 is delivered to a patient 310 intravenously. This blood access system allows for the automated calibration and periodic blood sampling of a patient's blood without operator intervention. Further, the system allows a single catheter 311 to be placed in the vein of a patient and blood sampled from this catheter 311. The system allows for direct measurement of blood analyte concentration, specifically glucose in the sampled blood. The system is able to perform periodic calibrations by exposing sensor 312 to known glucose concentration. This example embodiment has at least one calibration solution 300 and/or a flushing solution such as saline 302. Although in this embodiment the calibration solution is glucose and the flushing solution saline, those skilled in the art will recognize several alternatives to these solutions.

The calibration solution 300 and the saline 302 are connected to the fluid delivery pumps 304 and are connected to a calibration solution supply tube 352 and a saline supply tube 354 respectively. The calibration solution supply tube 352 has a check valve 351 before connecting to the saline supply tube 354 at the fluid tube junction 353. The fluid tube junction connects to a sample tube junction 320. Although this embodiment shows two separate pumps, those skilled in the art will recognize that multichannel pumps are available that can pump multiple tubes. Additionally, saline 302 is attached through the fluid supply tubes 306 but those skilled in the art recognize that saline 302 is optional. Further, those skilled in the art will recognize that syringe pumps, gravity pumps, or other devices are available to deliver a fluid through an IV. The fluid delivery pumps 304 are well known in the art and are capable of volumetric control of one or more IV tubes thereby controlling the delivery rate of one or more drugs. The fluid delivery pumps 304 are controllable from an electronic controller 336. Further, the fluid delivery pumps 304 may communicate with the controller, including communicating delivery status information.

A bidirectional patient tube 322 connects the sample tube junction 320 to the patient 310. More specifically, the bidirectional patient tube 322 terminates in a catheter 311 which is intravenously placed in the patient 310. Therefore, the fluid delivery pump 304 may pump calibration solution 300 through the fluid supply tube 306 and the bidirectional patient tube 322 to be infused intravenously into the patient 310. This example embodiment also contains an analyzer 312 which communicates with the controller 336. Those skilled in the art will recognize that the controller 336 may be external to the analyzer 312 as shown in FIG. 15 or may be incorporated within the analyzer 312. A sample tube 318 connects the analyzer 312 to the sample tube junction 320. The sample tube 318 has a clamp housing 316 and a blood dropper 328 at its terminus. Those skilled in the art will recognize that an indwelling biosensor or optical measurement method could be used as the sensor system contained in box 312.

When in use, the sample tube 318 extends from the sample tube junction 320 through the analyzer 312, with the clamp housing 316 set in the clamp housing holder 314 with a portion of the sample tube 318 extending around a peristaltic pump 324, occluding the sample tube 318. The peristaltic pump 324 is configured such that a platen arm 334 positions the sample tube 318 in compressioned contact with at least one roller 336 of the peristaltic pump 324. As the peristaltic pump 324 rotates the peristaltic pump 324 draws fluid through the sample tube 318 towards the analyzer, with the fluid being pushed out the blood dropper 328. The fluid supply tube 306, the bidirectional patient tube 322, and the sample tube 318 comprise the sample/supply set 308 shown in FIG. 16. The sample/supply set 308 of the preferred embodiment is a medical disposable unit that is used for a single use. That is, each patient serviced by the preferred embodiment requires a new, sterile sample/supply set 308. The sample/supply set 308 will be described in more detail below. Additional components of the preferred embodiment will be introduced and described while now presenting modes of operation. This example embodiment has four modes of operation: 1) infuse mode; 2) sample mode; 3) analyze mode; and 4) clear mode. Each mode is addressed separately below.

In mode one, infuse mode, the controller 336 instructs the fluid delivery pump 304 to pump calibration solution 300 into the calibration solution supply tube 352. The fluid flows from the calibration solution 300 fluid supply, through the calibration solution supply tube 352 toward the sample tube junction 320. In the infuse mode, the peristaltic pump 324 of the analyzer 312 is rotating so as to pull calibration solution into the sensor 312. Therefore, the calibration solution 300 flowing through the calibration solution supply tube 352 flows through the sample tube junction 320 into tubing 318 and into sensor 312. The system has the ability to deliver both calibration fluid and saline to the patient depending upon the activation of pump 334. Further, the saline 302 and calibration solution 300 can be infused individually or simultaneously. The rate of infusion is set by the controller. When the controller or the user indicates it is time to take and analyze a new sample, mode two begins.

In mode two, sample mode, the example embodiment first flushes the system. The calibration solution pump 304 is first turned off and the saline pump activated. Those actions pump saline 302 through the saline tube 354, sample tube junction 320, the bidirectional flush tube 322, and the catheter 311, and to the patient 310. Once the flush path from the fluid tube junction 353 to the patient 310 is cleared of calibration solution, the peristaltic pump 324 is activated at a rate somewhat less than the rate of the saline pump 304. Then, the peristaltic pump 324 draws saline from the sample tube junction 320 through the sample tube 318, and out the blood dropper 328, cleaning the sample tube 318 of calibration solution. Since the saline pump 304 is pumping a rate faster than the peristaltic pump, a small quantify of saline is still being pumped to the patient 310. With the system now cleared of calibration solution, all pumps are stopped. After flushing the system, the system can inflate the pressure cuff 307, which is placed around an extremity and near the intravenous entry point of the patient 310. As the intravenous entry point is often the arm or hand, the pressure cuff may be placed on the upper arm. It is known in the art that the pressure of an inflated pressure cuff assists in the drawing of blood. With the cuff inflated, the preferred embodiment now takes a fluid sample, generally blood, from the patient 310 and draws the blood sample to the analyzer 312. In this mode, the fluid delivery pump 304 is not operational so no calibration solution is being pumped through the fluid supply tube 306. The peristaltic pump 324 rotates (clockwise as illustrated) and as each roller 336 contacts the sample tube 318, that roller 336 pushes fluid in the sample tube toward the blood dropper 328. The blood dropper 328 is positioned on a blood dropper arm 340, which moves the blood dropper 328 from a standby area to a position above a waste container 338. With the blood dropper 328 positioned over the waste container 338, fluid pushed through the sample tube 318 is deposited in the waster container 338. As the peristaltic pump 324 turns, a bolus of blood is pulled from the patient and up the bidirectional patient tube 322 toward the analyzer 312.

As the peristaltic pump 324 continues to operate, the bolus of blood reaches the clamp housing 316. The clamp housing 316 may contain an optical sensor to detect the leading edge of the blood sample. Since the volume of fluid in the sample tube 318 after the clamp housing 316 is known, and the volume moved by the peristaltic pump 324 is also defined, it can be calculated how much the peristaltic pump 324 must rotate until the blood has reached the blood dropper 328. Alternatively, if no optical sensor is used, the peristaltic pump 324 is rotated a sufficient number of times to bring the blood from the patient 310 to the blood dropper 328. After the blood sample is at the blood dropper 328, the peristaltic pump 324 stops. As a drop of blood may be left hanging from the blood dropper, the peristaltic pump 324 can be rotated in the opposite direction a small amount to draw the blood drop back into the blood dropper 328. The blood dropper arm 340 now moves to place the blood dropper 328 over the test area 330. As the sample mode ends the pressure cuff is deflated.

Mode three, the analyze mode, now begins. The analyzer 312 has a slide cassette 332 which is vertically positionable. The slide cassette 332 holds several slides 346, with each slide having at least one area that has a reagent which reacts with a substance that may be present in the patient's blood sample. The cassette 332 is positioned vertically such that a slide arm 326 contacts the desired slide 346 in the cassette 332. The slide arm 326 pushes the slide 346 and positions it into the test area 330. The peristaltic pump 324 is activated for a short time, pushing a small amount of blood from the blood dropper 328 into the test area 330. As noted previously analyzer 312 can be replaced by a variety of sensor methods to include indwelling biosensors and optical measurement sensors.

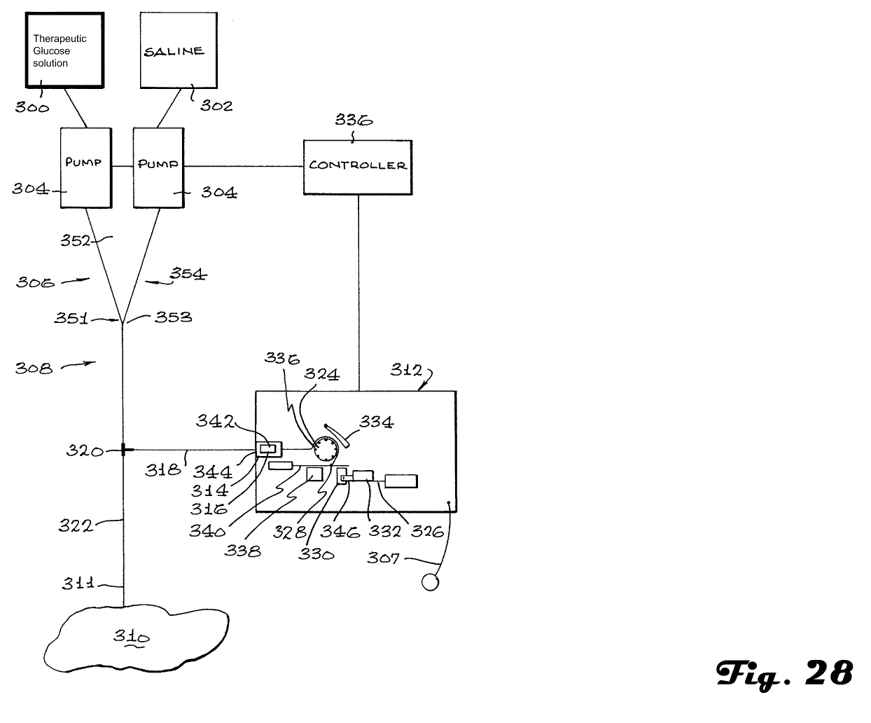

FIG. 28 is a schematic illustration of an example blood access system embodiment which also contains the ability to infuse therapeutic amounts of glucose for the effective implementation of tight glycemic control protocols. The therapeutic glucose solution can be insulin so as to decrease glucose concentrations or a solution containing glucose so as to increase the individual's glucose level. In this embodiment, a therapeutic glucose solution 300 is delivered to a patient 310 intravenously. This blood access system allows for the administration of a therapeutic glucose solution and periodic blood sampling of a patient's blood without operator intervention. Further, the system allows a single catheter 311 to be placed in the vein of a patient with the therapeutic glucose solution infused through this catheter 311 and at a later time, blood sampled from this same catheter 311. Such a system allows an operator to define a desired glucose level in a patient's bloodstream. The system allows for direct measurement of glucose concentration. For example, a preferred embodiment of the present system measures the glucose concentration thus indicating the need for the therapeutic glucose solution. At a predetermined time or variable time intervals, the system changes modes to allow blood to be sampled and tested. Based on the glucose level found in the bloodstream, the system adjusts the rate of the insulin or glucose infusion, thus titrating the glucose to the desired level. This example embodiment has at least one therapeutic solution such as therapeutic glucose solution 300 and/or a flushing solution such as saline 302. Although in this embodiment the therapeutic solution is glucose or insulin and the flushing solution saline, those skilled in the art will recognize alternatives to these solutions.

The therapeutic glucose solution 300 and the saline 302 are connected to the fluid delivery pumps 304 and are connected to a therapeutic glucose solution supply tube 352 and a saline supply tube 354 respectively. The therapeutic glucose solution supply tube 352 has a check valve 351 before connecting to the saline supply tube 354 at the fluid tube junction 353. The fluid tube junction connects to a sample tube junction 320. Although this embodiment shows two separate pumps, those skilled in the art will recognize that multichannel pumps are available that can pump multiple tubes. Additionally, saline 302 is attached through the fluid supply tubes 306 but those skilled in the art recognize that saline 302 is optional. Further, those skilled in the art will recognize that syringe pumps, gravity pumps, or other devices are available to deliver a fluid through an IV. The fluid delivery pumps 304 are well known in the art and are capable of volumetric control of one or more IV tubes thereby controlling the delivery rate of one or more drugs. The fluid delivery pumps 304 are controllable from an electronic controller 336. Further, the fluid delivery pumps 304 may communicate with the controller, including communicating delivery status information.

A bidirectional patient tube 322 connects the sample tube junction 320 to the patient 310. More specifically, the bidirectional patient tube 322 terminates in a catheter 311 which is intravenously placed in the patient 310. Therefore, the fluid delivery pump 304 may pump therapeutic glucose solution 300 through the fluid supply tube 306 and the bidirectional patient tube 322 to be infused intravenously into the patient 310. This example embodiment also contains an analyzer 312 which communicates with the controller 336. Those skilled in the art will recognize that the controller 336 may be external to the analyzer 312 as shown in FIG. 15 or may be incorporated within the analyzer 312. A sample tube 318 connects the analyzer 312 to the sample tube junction 320. The sample tube 318 has a clamp housing 316 and a blood dropper 328 at its terminus. Those skilled in the art will recognize that analyzer 312 can be a variety of glucose measurement methods to include biosensors and optical measurement systems.

When in use, the sample tube 318 extends from the sample tube junction 320 through the analyzer 312, with the clamp housing 316 set in the clamp housing holder 314 with a portion of the sample tube 318 extending around a peristaltic pump 324, occluding the sample tube 318. The peristaltic pump 324 is configured such that a platen arm 334 positions the sample tube 318 in compressioned contact with at least one roller 336 of the peristaltic pump 324. As the peristaltic pump 324 rotates the peristaltic pump 324 draws fluid through the sample tube 318 towards the analyzer, with the fluid being pushed out the blood dropper 328.

The fluid supply tube 306, the bidirectional patient tube 322, and the sample tube 318 comprise the sample/supply set 308 shown in FIG. 16. The sample/supply set 308 of the preferred embodiment is a medical disposable unit that is used for a single use. That is, each patient serviced by the preferred embodiment requires a new, sterile sample/supply set 308. The sample/supply set 308 will be described in more detail below. Additional components of the preferred embodiment will be introduced and described while now presenting modes of operation. This preferred embodiment has four modes of operation: 1) infuse mode; 2) sample mode; 3) analyze mode; and 4) clear mode. Each mode is addressed separately below.

In mode one, infuse mode, the controller 336 instructs the fluid delivery pump 304 to pump therapeutic glucose solution 300 into the therapeutic glucose solution supply tube 352. The fluid flows from the therapeutic glucose solution 300 fluid supply, through the therapeutic glucose solution supply tube 352 toward the sample tube junction 320. In the infuse mode, the peristaltic pump 324 of the analyzer 312 is not rotating and the platen arm 334 is pressed tightly against one or more of the rollers 336, thus occluding the sample tube 318. Therefore, the therapeutic glucose solution 300 flowing through the therapeutic glucose solution supply tube 352 flows through the sample tube junction 320 into the bidirectional patient tube 322 and via the catheter 311 into the patient 310. In a similar manner, the preferred embodiment may deliver saline 302 to the patient 310. Further, the saline 302 and therapeutic glucose solution 300 can be infused individually or simultaneously. The rate of infusion is set by the controller. When the controller or the user indicates it is time to take and analyze a new sample, mode two begins.

In mode two, sample mode, the system first flushes the system. The therapeutic glucose solution pump 304 is first turned off and the saline pump activated. Those actions pump saline 302 through the saline tube 354, sample tube junction 320, the bidirectional flush tube 322, and the catheter 311, and to the patient 310. Once the flush path from the fluid tube junction 353 to the patient 310 is cleared of therapeutic glucose solution, the peristaltic pump 324 is activated at a rate somewhat less than the rate of the saline pump 304. Then, the peristaltic pump 324 draws saline from the sample tube junction 320 through the sample tube 318, and out the blood dropper 328, cleaning the sample tube 318 of therapeutic glucose solution. Since the saline pump 304 is pumping a rate faster than the peristaltic pump, a small quantify of saline is still being pumped to the patient 310. With the system now cleared of therapeutic glucose solution, all pumps are stopped.

After flushing the system, the system may inflate the pressure cuff 307, which is placed around an extremity and near the intravenous entry point of the patient 310. As the intravenous entry point is often the arm or hand, the pressure cuff may be placed on the upper arm. It is known in the art that the pressure of an inflated pressure cuff assists in the drawing of blood. With the cuff inflated, the preferred embodiment now takes a fluid sample, generally blood, from the patient 310 and draws the blood sample to the analyzer 312. In this mode, the fluid delivery pump 304 is not operational so no therapeutic solution is being pumped through the fluid supply tube 306. The peristaltic pump 324 rotates (clockwise as illustrated) and as each roller 336 contacts the sample tube 318, that roller 336 pushes fluid in the sample tube toward the blood dropper 328. The blood dropper 328 is positioned on a blood dropper arm 340, which moves the blood dropper 328 from a standby area to a position above a waste container 338. With the blood dropper 328 positioned over the waste container 338, fluid pushed through the sample tube 318 is deposited in the waster container 338. As the peristaltic pump 324 turns, a bolus of blood is pulled from the patient and up the bidirectional patient tube 322 toward the analyzer 312.

As the peristaltic pump 324 continues to operate, the bolus of blood reaches the clamp housing 316. The clamp housing 316 may contain an optical sensor to detect the leading edge of the blood sample. Since the volume of fluid in the sample tube 318 after the clamp housing 316 is known, and the volume moved by the peristaltic pump 324 is also defined, it can be calculated how much the peristaltic pump 324 must rotate until the blood has reached the blood dropper 328. Alternatively, if no optical sensor is used, the peristaltic pump 324 is rotated a sufficient number of times to bring the blood from the patient 310 to the blood dropper 328. After the blood sample is at the blood dropper 328, the peristaltic pump 324 stops. As a drop of blood may be left hanging from the blood dropper, the peristaltic pump 324 can be rotated in the opposite direction a small amount to draw the blood drop back into the blood dropper 328. The blood dropper arm 340 now moves to place the blood dropper 328 over the test area 330. As the sample mode ends the pressure cuff is deflated.

Mode three, the analyze mode, now begins. The analyzer 312 has a slide cassette 332 which is vertically positionable. The slide cassette 332 holds several slides 346, with each slide having at least one area that has a reagent which reacts with a substance that may be present in the patient's blood sample. The cassette 332 is positioned vertically such that a slide arm 326 contacts the desired slide 346 in the cassette 332. The slide arm 326 pushes the slide 346 and positions it into the test area 330. The peristaltic pump 324 is activated for a short time, pushing a small amount of blood from the blood dropper 328 into the test area 330.

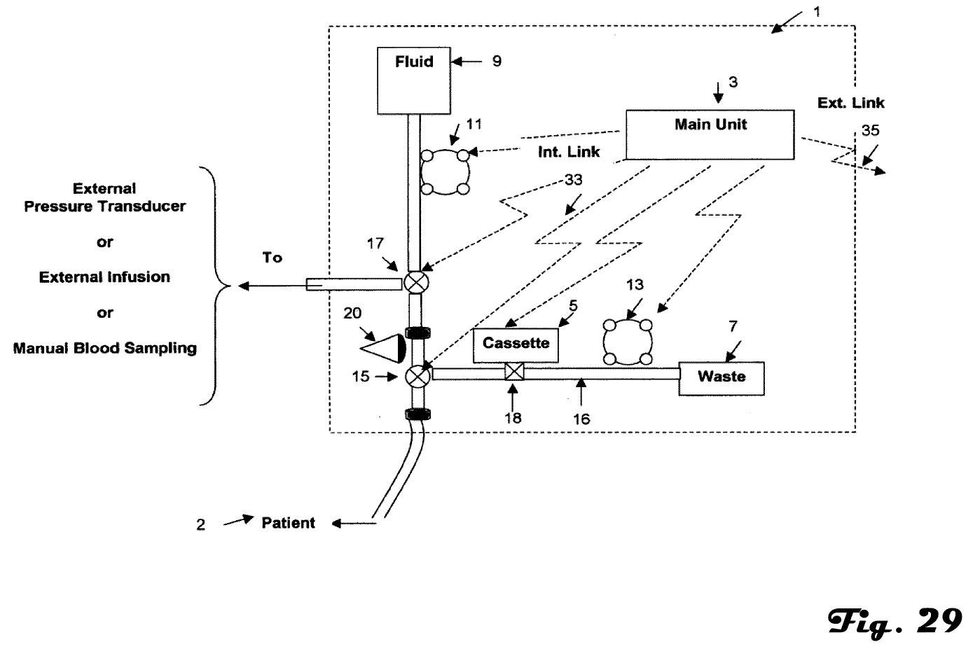

FIG. 29 is a schematic illustration of a layout of the functional elements of an example embodiment of an automated device for analyzing blood parameters. As shown in FIG. 29, automated blood analysis device 1 is a device for automatically measuring blood analytes and blood parameters. Automated blood analysis device 1 is connected to a catheter leading to the patient 2, in order to automatically collect blood samples and automatically measure required blood parameters. Preferably, automated blood analysis device 1 comprises main unit 3; sensor cassette 5, which is preferably disposable; waste container 7; fluid container 9; first infusion pump 11; and second infusion pump 13. The first infusion pump 11 and second infusion pump 13 are volumetric infusion pumps as are well-known in the art for use in intravenous fluid administration systems, although other types of pumps such as peristaltic pumps, piston pumps, or syringe pumps can also be used. Also, but not limited to such uses, it is preferred that first infusion pump 11 is used to control the flow in the fluid delivery line from fluid container 9 and second infusion pump 13 is used to control the flow in line 16 used for drawing blood samples to sensor cassette 5.

Automated blood analysis device 1 also comprises a series of tubes, including line 16, which are described in further detail below. In addition, automated blood analysis device 1 includes a first automated three-way stopcock 15 for controlling the flow inside line 16 and a second automated three-way stopcock 17 for controlling the flow of fluids to and from the external tubing and/or external devices. The operation of first stopcock 15 and second stopcock 17 is preferably fully automated and controlled by main unit 3. An automated sampling interface mechanism 18, described in further detail below, enables a blood sample to be brought automatically from line 16 to sensor 19 within sensor cassette 5. one of skill in the art will recognize that sensor cassette 5 can be replaced by a variety of glucose measurement means to include a biosensor in contact with the fluid and an optical measurement system. As further described in detail, automated blood analysis device 1 can work as a stand-alone device, or can be connected in parallel with external infusions (on the same venous line) or external pressure transducers (on the same arterial line) a location of connectivity is shown in FIG. 29. Automated blood analysis device 1 enables blood sampling and analysis on demand.

In the example embodiment of FIG. 29, the operational steps of automated blood analysis device 1 are described according to a preferred workflow when automated blood analysis device 1 is connected in parallel to external infusions at the same vascular access point. It is to be understood that such embodiment is exemplary but not limiting and that the automated blood analysis device 1 may be connected to other external devices at the same vascular access point. Automated blood analysis device 1 blocks the operation of any connected infusion and/or external device (such as an external pressure transducer) during the period of blood sampling, in order to ensure that the blood sample is not diluted/altered by other fluids injected in the patient. During normal operation, first stopcock 15 blocks line 16 and keeps the line to patient 2 open and second stopcock 17 enables the external infusion to flow freely into patient 2 while at the same time blocking the line coming from fluid bag 9.

When performing automated blood sampling and measurement of required blood analytes, main unit 3 directs second stopcock 17 to block incoming external infusions and to open the line from fluid bag 9 to patient 2. Once the external infusions are interrupted, pump 11 draws blood from patient 2. The blood is drawn along the tube until the remaining infusion volume and the initially diluted blood volume passes first stopcock 15. Main unit 3 calculates the required volume of blood to be withdrawn based on the diameter and length of the tubing and according to a programmable dead-space volume, which can be either pre-calibrated or user-defined. Optionally, a blood optical sensor 20 can be used to establish whether undiluted blood has reached the tube segment proximal to first stopcock 15. When undiluted blood reaches first stopcock 15, first stopcock 15 is repositioned to create an open line between patient 2 and sensor cassette 5. Blood is then pumped into line 16 via pump 13.

When undiluted blood reaches the tube segment proximal to sensor cassette 5, a blood sample is automatically taken inside sensor cassette 5 (by sampling interface mechanism 18) whereby a sensor 19 (from a plurality of sensors within sensor cassette 5) is placed into contact with the drawn blood sample. Sensor 19 is preferably, but not limited to, a single use sensor, and is used to measure patient blood analyte(s) and blood parameter(s). Sensor 19 is preferably a component of a manual test device, such as, but not limited to glucose test strips for measuring glucose levels. While the blood sample is analyzed, blood withdrawal from patient 2 is stopped, main unit 3 reverses the operation of pump 11, and first stopcock 15 is repositioned to infuse blood back into patient 2. The tubing components, including line 16, are then flushed by purging fluid from fluid bag 9. Blood and fluids from line 16 are stored in waste container 7, which is, for example, but not limited to a waste bag generally used for storage of biological disposals. Optionally, the remaining blood in line 16 can be infused back into patient 2 by reversing the direction of pump 13. After purging both line 16 and the line between fluid bag 9 and patient 2, main unit 3 redirects first stopcock 15 and second stopcock 17 to block both line 16 and the line between fluid bag 9 and patient 2 and reopen the line from the external infusion device, into patient 2.

Referring back to FIG. 29 in an alternate workflow of an example embodiment of the present invention, once enough blood is withdrawn and pumped to line 16, stopcock 15 is turned and the volume of blood in line 16 is pushed by the fluid coming from fluid bag 9. This method is referred to as using a “bolus of blood” and is designed to reduce the amount of blood withdrawn in line 16. The remaining steps in this alternate workflow are as described above with respect to the example embodiment in FIG. 29.

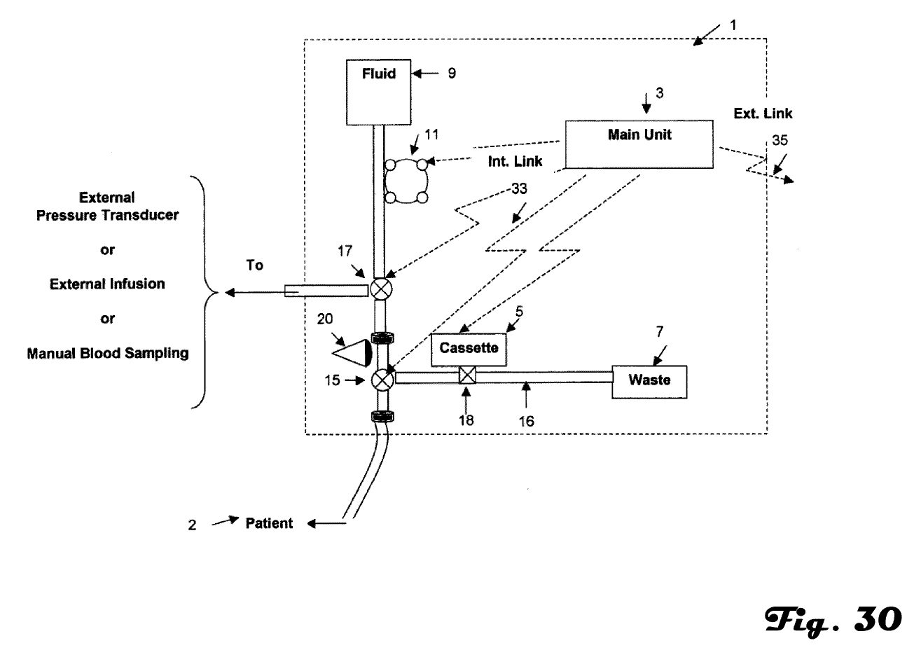

FIG. 30 is a schematic illustration of the layout of the functional elements and workflow of an example embodiment of an automated blood analysis device. This embodiment will be described with reference to FIG. 8, noting the differences between the designs. In the second preferred embodiment, automated blood analysis device 1 employs a single pump 11 and does not require the usage of second pump 13 (as shown in FIG. 8). Operationally, an extra dead-space volume is initially withdrawn by single pump 11 to ensure that an undiluted blood volume has passed stopcock 15. When the undiluted blood volume passes stopcock 15, stopcock 15 is repositioned to create an open line between pump 11 and sensor cassette 5. The undiluted blood volume is then pushed into line 16 by pump 11. The remaining operational steps are not modified with respect to the embodiment illustrated in FIG. 8, and thus will not be repeated herein.

FIG. 31 is a schematic illustration of the layout of the functional elements and workflow of an example embodiment of a blood analysis device. Again, this embodiment will be described with reference to the previous figures, noting the differences between the designs. In the fourth preferred embodiment of the blood analysis device of the present invention, the device comprises a single pump 11, two additional stopcocks 26 and 27, and line 28 positioned between stopcock 26 and stopcock 15. The operation of the fourth embodiment is described in further detail below. In order to withdraw blood into line 16, stopcock 15 is turned to block the main tube and blood is withdrawn above stopcock 27 by pump 11. Once the blood is drawn above stopcock 27, stopcock 27 is turned while the operation of pump 11 is reversed, thus pushing blood through stopcock 27 into line 16. The blood in the line is then flushed with purging fluid from fluid container 9. Stopcock 27 is then turned again, thus enabling infusion back into line 28. Now referring back to FIGS. 29-31, the infusion tube and line 16, as used in the first and second embodiments 9 and 9, respectively, can be made of commonly used flexible transparent plastic materials such as polyurethane, silicone or PVC. When line 16 is present in any particular embodiment, it is preferably of the smallest diameter possible, while still enabling blood flow without clotting or hemolysis. For example, and not limited to such example, line 16 has a diameter of less than or equal to 1 mm.

The tubing and stopcocks/valve sets of the example embodiments can be implemented in various designs to support operational requirements. Optionally, the tubing includes filter lines to enable elimination of air embolism and particle infusion. Additionally, the tubing can optionally include a three-way stopcock that enables the user/clinician to manually draw blood samples for laboratory tests. In addition, three-way stopcock 17 may optionally include a plurality of stopcocks at its inlet, each controlling a separate external line. In another optional embodiment, the positions of stopcock 15 and stopcock 17 can be interchanged, thus placing stopcock 17 closer to the vascular access point in patient 2 than stopcock 15 or cassette 5. Automated blood analysis device 1 is connected to an insertion element, such as, but not limited to a catheter or a Venflon (not shown), inserted into a vein or artery to provide a flow path for fluid infusion and drawing of patient blood samples. Insertion into a vein or artery is performed according to existing clinical indications that are well known to those of ordinary skill in the art. This design avoids repeated insertions of needles or catheter structures into the patient as is commonly required with prior art blood chemistry monitoring techniques. Connection of the automated blood analysis device 1 to the catheter or venflon is made by standard means such as luer-lock connectors, as are known in the art. Optionally, the insertion element, catheter or venflon, can be part of the tubing of automated device for analyzing blood 1. In another example embodiment, the catheter may comprise a multi-lumen catheter wherein one of the lumens is used for automatically drawing the blood sample.

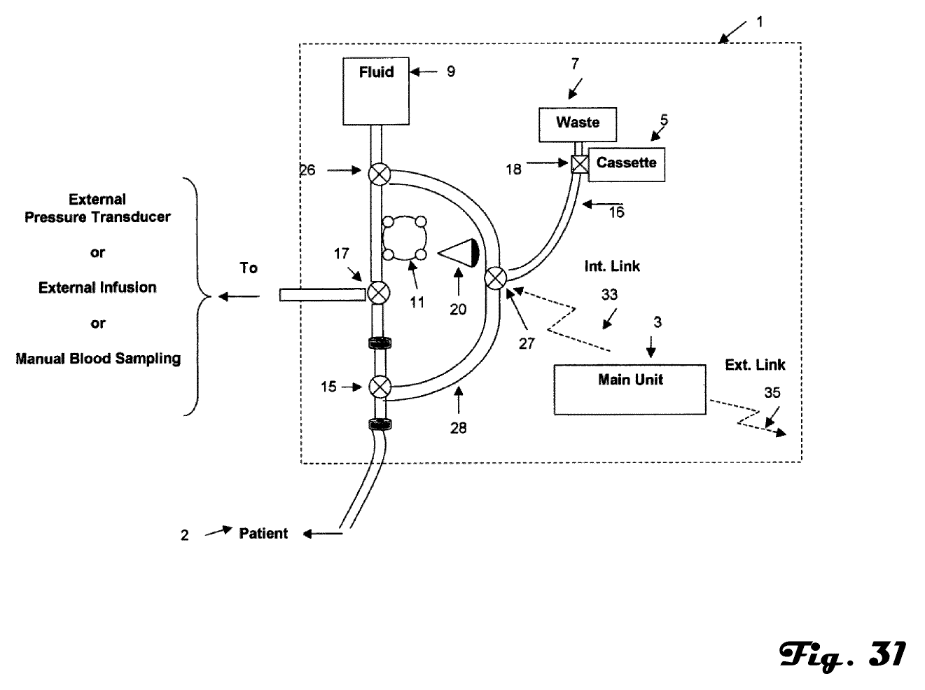

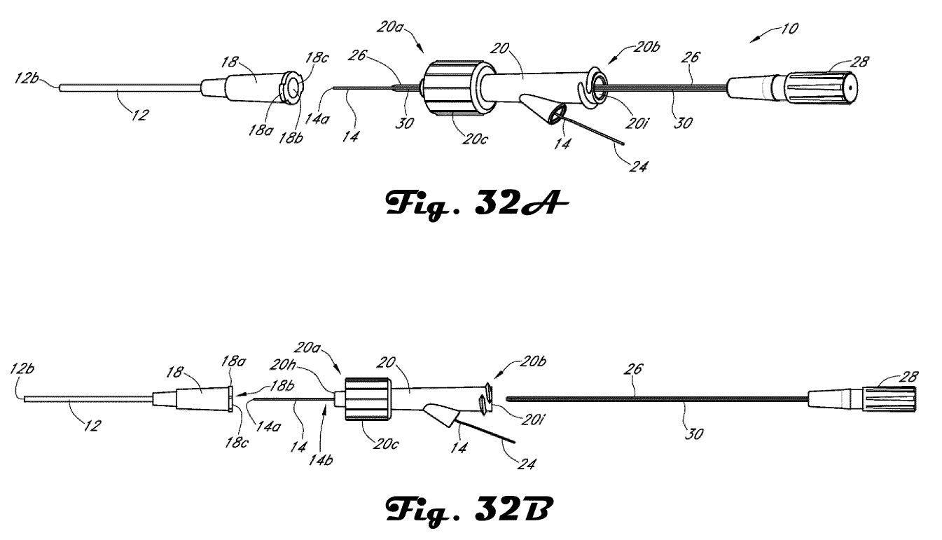

FIGS. 32A and 32B illustrate an example embodiment of a blood access and measurement system. The analyte sensor system 10 includes a catheter 12 configured to be inserted or pre-inserted into a host's blood stream. In clinical settings, catheters are often inserted into hosts to allow direct access to the circulatory system without frequent needle insertion (e.g., venipuncture). Suitable catheters can be sized as is known and appreciated by one skilled in the art, such as but not limited to from about 1 French (0.33 mm) or less to about 30 French (10 mm) or more; and can be, for example, 2, 3, 4, 5, 6, 7, 8, 9, 10, 11, 12, 13, 14, 15, 16, 17, 18, 19, or 20 French (3 French is equivalent to about 1 mm) and/or from about 33 gauge or less to about 16 gauge or more, for example, 33, 32, 31, 30, 29, 28, 27, 26, 25, 24, 23, 22, 21, 20, 19, 18, 17, or 16 gauge. Additionally, the catheter can be shorter or longer, for example 0.75, 1.0, 1.25, 1.5, 1.75, 2.0 inches in length or longer. In some embodiments, the catheter is a venous catheter. In other embodiments, the catheter is configured for insertion into a peripheral or a central artery. In some embodiments, the catheter is configured to extend from a peripheral artery to a central portion of the host's circulatory system, such as but not limited to the heart. The catheter can be manufactured of any medical grade material known in the art, such as but not limited to polymers and glass as described herein. A catheter can include a single lumen or multiple lumens. A catheter can include one or more perforations, to allow the passage of host fluid through the lumen of the catheter.

The terms “inserted” or “pre-inserted” as used herein are broad terms, and are to be given their ordinary and customary meaning to a person of ordinary skill in the art (and are not to be limited to a special or customized meaning), and refer without limitation to insertion of one thing into another thing. For example, a catheter can be inserted into a host's blood stream. In some embodiments, a catheter is “pre-inserted,” meaning inserted before another action is taken (e.g., insertion of a catheter into a host's blood stream prior to insertion of a sensor into the catheter). In some exemplary embodiments, a sensor is coupled to a pre-inserted catheter, namely, one that has been previously inserted (or pre-inserted) into the host's circulatory system.

Referring now to FIGS. 32A and 32B, the catheter 12 is a thin, flexible tube having a lumen 12a, such as is known in the art. In some embodiments, the catheter can be rigid; in other embodiments, the catheter can be custom manufactured to desired specifications (e.g., rigidity, dimensions, etc). The catheter can be a single-lumen catheter or a multi-lumen catheter. At the catheter's proximal end is a small orifice 12b for fluid connection of the catheter to the blood stream. At the catheter's distal end is a connector 18, such as a leur connector or other fluid connector known in the art.

FIGS. 32A and 32B show one exemplary embodiment of the connector 18 including a flange 18a and a duct 18b. In the exemplary embodiment, the flange 18a is configured to enable connection of the catheter to other medical equipment (e.g., saline bag, pressure transducer, blood chemistry device, and the like) or capping (e.g., with a bung and the like). Although one exemplary connector is shown, one skilled in the art appreciates a variety of standard or custom made connectors suitable for use with the preferred embodiments. The duct 18b is in fluid communication with the catheter lumen and terminates in a connector orifice 18c.

In some embodiments, the catheter is inserted into the host's blood stream, such as into a vein or artery by any useful method known in the art. Generally, prior to and during insertion, the catheter is supported by a hollow needle or trochar (not shown). For example, the supported catheter can be inserted into a peripheral vein or artery, such as in the host's arm, leg, hand, or foot. Typically, the supporting needle is removed (e.g., pulled out of the connector) and the catheter is connected (e.g., via the connector 18) to IV tubing and a saline drip, for example. However, in one embodiment, the catheter is configured to operatively couple to medical equipment, such as but not limited to a sensor system of the preferred embodiments. Additionally and/or alternatively, the catheter can be configured to operatively couple to another medical device, such as a pressure transducer, for measurement of the host's blood pressure.

In some embodiments, the catheter and the analyte sensor are configured to indwell within the host's blood stream in vivo. An indwelling medical device, such as a catheter or implant, is disposed within a portion of the body for a period of time, from a few minutes or hours to a few days, months, or even years. In some embodiments, the catheter can indwell in a host's artery or vein for the length of a perioperative period (e.g., the entire hospital stay) or for shorter or longer periods. In some embodiments, the use of an indwelling catheter permits continuous access of an analyte sensor to a blood stream while simultaneously allowing continuous access to the host's blood stream for other purposes, for example, the administration of therapeutics (e.g., fluids, drugs, etc.), measurement of physiologic properties (e.g., blood pressure), fluid removal, and the like.

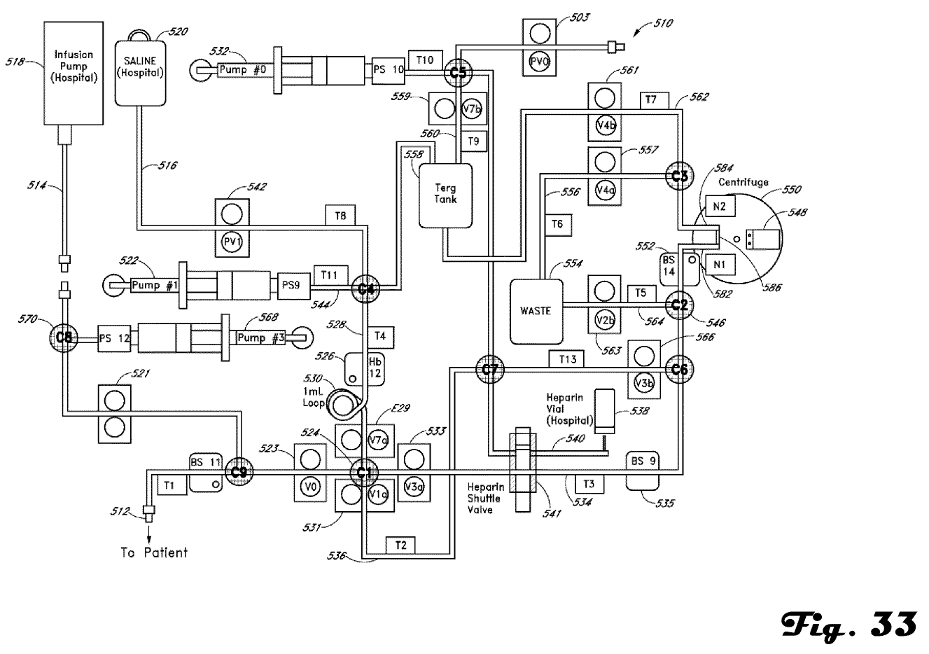

FIG. 33 illustrates another example blood access system that can be used for automated blood glucose monitoring. The fluid system 510 is described practically to show an example cycle as fluid is drawn and analyzed. In addition to the reference numerals used below, the various portions of the illustrated fluid system 510 are labeled for convenience with letters to suggest their roles as follows: T# indicates a section of tubing. C# indicates a connector that joins multiple tubing sections. V# indicates a valve. BS# indicates a bubble sensor or ultrasonic air detector. N# indicates a needle (e.g., a needle that injects sample into a sample holder). PS# indicates a pressure sensor (e.g., a reusable pressure sensor). Pump# indicates a fluid pump (e.g., a syringe pump with a disposable body and reusable drive). “Hb 12” indicates a sensor for hemoglobin (e.g., a dilution sensor that can detect hemoglobin optically).

The function of the valves, pumps, actuators, drivers, motors (e.g., the centrifuge motor), etc. described below is controlled by one or more controllers (e.g., the fluid system controller 405, the optical system controller 413, etc.) The controllers can include software, computer memory, electrical and mechanical connections to the controlled components, etc. At the start of a measurement cycle, most lines, including a patient tube 512 (T1), an Hb sensor tube 528 (T4), an anticoagulant valve tube 534 (T3), and a sample cell 548 can be filled with saline that can be introduced into the system through the infusion tube 514 and the saline tube 516, and which can come from an infusion pump 518 and/or a saline bag 520. The infusion pump 518 and the saline bag 520 can be provided separately from the system 510. For example, a hospital can use existing saline bags and infusion pumps to interface with the described system. The infusion valve 521 can be open to allow saline to flow into the tube 512 (T1).

Before drawing a sample, the saline in part of the system 510 can be replaced with air. Thus, for example, the following valves can be closed: air valve 503 (PV0), the terg tank valve 559 (V7b), 566 (V3b), 523 (V0), 529 (V7a), and 563 (V2b). At the same time, the following valves can be open: valves 531 (V1a), 533 (V3a) and 577 (V4a). Simultaneously, a second pump 532 (pump #0) pumps air through system 510, pushing saline through tube 534 (T3) and sample cell 548 into a waste bladder 554.

Next, a sample can be drawn. With the valves 542 (PV1), 559 (V7b), and 561 (V4b) closed, a first pump 522 (pump #1) is actuated to draw sample fluid to be analyzed (e.g. blood) from a fluid source (e.g., a laboratory sample container, a living patient, etc.) up into the patient tube 512 (T1), through the tube past the two flanking portions of the open pinch-valve 523 (V0), through the first connector 524 (C1), into the looped tube 530, past the hemoglobin sensor 526 (Hb12), and into the Hb sensor tube 528 (T4). During this process, the valve 529 (V7a) and 523 (V0) are open to fluid flow, and the valves 531 (V1a), 533 (V3a), *42 (PV1), *59 (V7b), and 561 (V4b) can be closed and therefore block (or substantially block) fluid flow by pinching the tube.

Before drawing the sample, the tubes 512 (T1) and 528 (T4) are filled with saline and the hemoglobin (Hb) level is zero. The tubes that are filled with saline are in fluid communication with the sample source (e.g., the fluid source 402). The sample source can be the vessels of a living human or a pool of liquid in a laboratory sample container, for example. When the saline is drawn toward the first pump 522, fluid to be analyzed is also drawn into the system because of the suction forces in the closed fluid system. Thus, the first pump 522 draws a relatively continuous column of fluid that first comprises generally nondiluted saline, then a mixture of saline and sample fluid (e.g., blood), and then eventually nondiluted sample fluid. In the example illustrated here, the sample fluid is blood.

The hemoglobin sensor 526 (Hb12) detects the level of Hemoglobin in the sample fluid. As blood starts to arrive at the hemoglobin sensor 526 (Hb12), the hemoglobin level rises. A hemoglobin level can be selected, and the system can be pre-set to determine when that level is reached. A controller such as the fluid system controller 405 of FIG. 4 can be used to set and react to the pre-set value, for example. In some embodiments, when the sensed hemoglobin level reaches the pre-set value, substantially undiluted sample is present at the first connector 524 (C1). The preset value can depend, in part, on the length and diameter of any tubes and/or passages traversed by the sample. In some embodiments, the pre-set value can be reached after approximately 2 mL of fluid (e.g., blood) has been drawn from a fluid source. A nondiluted sample can be, for example, a blood sample that is not diluted with saline solution, but instead has the characteristics of the rest of the blood flowing through a patient's body. A loop of tubing 530 (e.g., a 1-mL loop) can be advantageously positioned as illustrated to help insure that undiluted fluid (e.g., undiluted blood) is present at the first connector 524 (C1) when the hemoglobin sensor 526 registers that the preset Hb threshold is crossed. The loop of tubing 530 provides additional length to the Hb sensor tube 528 (T4) to make it less likely that the portion of the fluid column in the tubing at the first connector 524 (C1) has advanced all the way past the mixture of saline and sample fluid, and the nondiluted blood portion of that fluid has reached the first connector 524 (C1).

In some embodiments, when nondiluted blood is present at the first connector 524 (C1), a sample is mixed with an anticoagulant and is directed toward the sample cell 548. An amount of anticoagulant (e.g., heparin) can be introduced into the tube 534 (T3), and then the undiluted blood is mixed with the anticoagulant. A heparin vial 538 (e.g., an insertable vial provided independently by the user of the system 510) can be connected to a tube 540. An anticoagulant valve 541 (which can be a shuttle valve, for example) can be configured to connect to both the tube 540 and the anticoagulant valve tube 534 (T3). The valve can open the tube 540 to a suction force (e.g., created by the pump 532), allowing heparin to be drawn from the vial 538 into the valve 541. Then, the anticoagulant valve 541 can slide the heparin over into fluid communication with the anticoagulant valve tube 534 (T3). The anticoagulant valve 541 can then return to its previous position. Thus, heparin can be shuttled from the tube 540 into the anticoagulant valve tube 534 (T3) to provide a controlled amount of heparin into the tube 534 (T3).

With the valves 542 (PV1), 559 (V7b), 561 (V4b), 523 (V0), 531 (V1a), 566 (V3b), and 563 (V2b) closed, and the valves 529 (V7a) and 553 (V3a) open, first pump 522 (pump #1) pushes the sample from tube 528 (T4) into tube 534 (T3), where the sample mixes with the heparin injected by the anticoagulant valve 541 as it flows through the system 510. The sample continues to flow until a bubble sensor 535 (B S9) indicates the presences of the bubble. In some embodiments, the volume of tube 534 (T3) from connector 524 (C1) to bubble sensor 535 (BS9) is a known amount, and may be, for example, approximately 100 microliters.

When bubble sensor 535 (BS9) indicates the presence of a sample, the remainder of the sampled blood can be returned to its source (e.g., the patient veins or arteries). The first pump 522 (pump #1) pushes the blood out of the Hb sensor tube 528 (T4) and back to the patient by opening the valve 523 (V0), closing the valves 531 (V1a) and 533 (V3a), and keeping the valve 529 (V7a) open. The Hb sensor tube 528 (T4) is preferably flushed with approximately 2 mL of saline. This can be accomplished by closing the valve 529 (V7a), opening the valve 542 (PV1), drawing saline from the saline source 520 into the tube 544, closing the valve 542 (PV1), opening the valve 529 (V7a), and forcing the saline down the Hb sensor tube 528 (T4) with the pump 522. In some embodiments, less than two minutes elapse between the time that blood is drawn from the patient and the time that the blood is returned to the patient.

Following return of the unused patient blood sample, the sample is pushed up the anticoagulant valve tube 534 (T3), through the second connector 546 (C2), and into the sample cell 548, which can be located on the centrifuge rotor 550. This fluid movement is facilitated by the coordinated action (either pushing or drawing fluid) of the pump E22 (pump #1), the pump E32 (pump #0), and the various illustrated valves. Pump movement and valve position corresponding to each stage of fluid movement can be coordinated by one or multiple controllers, such as a fluid system controller.

After the unused sample is returned to the patient, the sample can be divided into separate slugs before being delivered into the sample cell 548. Thus, for example, valves 553 (V3a) and 531 (V1a) are opened, valves 523 (V0) and 529 (V7a) are closed, and the first pump 522 (pump #1) uses saline to push the sample towards sample cell 548. In some embodiments, the sample (for example 100 microliters) is divided into four “slugs” of sample, each separated by a small amount of air. As used herein, the term “slug” refers to a continuous column of fluid that can be relatively short. Slugs can be separated from one another by small amounts of air (or bubbles) that can be present at intervals in the tube. In some embodiments, the slugs are formed by injecting or drawing air into fluid in the first connector 546 (C2).

In some embodiments, when the leading edge of the sample reaches blood sensor 553 (BS14), a small amount of air (the first “bubble”) is injected at a connector 546 (C2), defining the first slug, which extends from the bubble sensor to the first bubble. In some embodiments, the valves 503 (PV0) and 559 (V7b) are closed, the valve 556 (V3b) is open, the pump 532 is actuated briefly to inject a first air bubble into the sample, and then valve 556 (V3b) is closed.

In some embodiments, the volume of the tube 534 (T3) from the connector 546 (C2) to the bubble sensor 552 (BS14) is less than the volume of tube 534 (T3) from the connector 524 (C1) to the bubble sensor 535 (BS9). Thus, for example and without limitation, the volume of the tube 534 (T3) from the connector 524 (C1) to the bubble sensor 535 (BS9) is approximately 100 .mu.L, and the volume of the tube 534 (T3) from the connector 546 (C2) to the bubble sensor 552 (BS14) is approximately 15 .mu.L. In some embodiments, four blood slugs are created. The first three blood slugs can have a volume of approximately 15 .mu.L and the fourth can have a volume of approximately 35 .mu.L.

A second slug can be prepared by opening the valves 553 (V3a) and 531 (V1a), closing the valves 523 (V0) and 529 (V7a), and operating the first pump 522 (pump #1) to push the first slug through a first sample cell holder interface tube 582 (N1), through the sample cell 548, through a second sample cell holder interface tube 584 (N2), and toward the waste bladder 554. When the first bubble reaches the bubble sensor 552 (BS14), the first pump 522 (pump #1) is stopped, and a second bubble is injected into the sample, as before. A third slug can be prepared in the same manner as the second (pushing the second bubble to bubble sensor 552 (BS14) and injecting a third bubble). After the injection of the third air bubble, the sample can be pushed through system 510 until the end of the sample is detected by bubble sensor 552 (BS14). The system can be designed such that when the end of the sample reaches this point, the last portion of the sample (a fourth slug) is within the sample cell 548, and the pump 522 can stop forcing the fluid column through the anticoagulant valve tube 534 (T3) so that the fourth slug remains within the sample cell 548. Thus, the first three blood slugs can serve to flush any residual saline out the sample cell 548. The three leading slugs can be deposited in the waste bladder 554 by passing through the tube F56 (T6) and past the tube-flanking portions of the open pinch valve 557 (V4a).

In some embodiments, the fourth blood slug is centrifuged for two minutes at 7200 RPM. Thus, for example, the sample cell holder interface tubes 582 (N1) and 584 (N2) disconnect the sample cell 548 from the tubes 534 (T3) and 562 (T7), permitting the centrifuge rotor 550 and the sample cell 548 to spin together. Spinning separates a sample (e.g., blood) into its components, isolates the plasma, and positions the plasma in the sample cell 548 for measurement. The centrifuge 550 can be stopped with the sample cell 548 in a beam of radiation (not shown) for analysis. The radiation, a detector, and logic can be used to analyze the a portion of the sample (e.g., the plasma) spectroscopically (e.g., for glucose, lactate, or other analyte concentration).

In some embodiments, portions of the system 510 that contain blood after the sample cell 548 has been provided with a sample are cleaned to prevent blood from clotting. Accordingly, the centrifuge rotor 550 can include two passageways for fluid that may be connected to the sample cell holder interface tubes 582 (N1) and 584 (N2). One passageway is sample cell 548, and a second passageway is a shunt 586. An embodiment of the shunt 586 is illustrated in more detail in FIG. 10B.

The shunt 586 can allow cleaner (e.g., tergazyme A) to flow through and clean the sample cell holder interface tubes without flowing through the sample cell 548. After the sample cell 548 is provided with a sample, the interface tubes 582 (N1) and 584 (N2) are disconnected from the sample cell 548, the centrifuge rotor 550 is rotated to align the shunt 586 with the interface tubes 582 (N1) and 584 (N2), and the interface tubes are connected with the shunt. With the shunt in place, the terg tank 559 is pressurized by the second pump 532 (pump #0) with valves 561 (V4b) and 563 (V2b) open and valves 557 (V4a) and 533 (V3a) closed to flush the cleaning solution back through the interface tubes 582 (N1) and 584 (N2) and into the waste bladder 554. Subsequently, saline can be drawn from the saline bag 520 for a saline flush. This flush pushes saline through the Hb sensor tube 528 (T4), the anticoagulant valve tube 534 (T3), the sample cell 548, and the waste tube 556 (T6). Thus, in some embodiments, the following valves are open for this flush: 529 (V7a), 533 (V3a), 557 (V4a), and the following valves are closed: 542 (PV1), 523 (V0), 531 (V1a), 566 (V3b), 563 (V2b), and 561 (V4b).

Following analysis, the second pump 532 (pump #0) flushes the sample cell 548 and sends the flushed contents to the waste bladder 554. This flush can be done with a cleaning solution from the terg tank 558. In some embodiments, the second pump 532 is in fluid communication with the terg tank tube 560 (T9) and the terg tank 558 because the terg tank valve 559 (V7b) is open. The second pump 532 forces cleaning solution from the terg tank 558 between the tube-flanking portions of the open pinch valve 561 and through the tube 562 (T7) when the valve 559 is open. The cleaning flush can pass through the sample cell 548, through the second connector 546, through the tube 564 (T5) and the open valve 563 (V2b), and into the waste bladder 554.

Subsequently, the first pump 522 (pump #1) can flush the cleaning solution out of the sample cell 548 using saline in drawn from the saline bag 520. This flush pushes saline through the Hb sensor tube 528 (T4), the anticoagulant valve tube 534 (T3), the sample cell 548, and the waste tube 556 (T6). Thus, in some embodiments, the following valves are open for this flush: 529 (V7a), 533 (V3a), 557 (V4a), and the following valves are closed: 542 (PV1), 523 (V0), 531 (V1a), 566 (V3b), 563 (V2b), and 561 (V4b).

When the fluid source is a living entity such as a patient, a low flow of saline (e.g., 1-5 mL/hr) is preferably moved through the patient tube 512 (T1) and into the patient to keep the patient's vessel open (e.g., to establish a keep vessel open, or “KVO” flow). This KVO flow can be temporarily interrupted when fluid is drawn into the fluid system 510. The source of this KVO flow can be the infusion pump 518, the third pump 568 (pump #3), or the first pump 522 (pump #1). In some embodiments, the infusion pump 518 can run continuously throughout the measurement cycle described above. This continuous flow can advantageously avoid any alarms that may be triggered if the infusion pump 518 senses that the flow has stopped or changed in some other way. In some embodiments, when the infusion valve 521 closes to allow pump 522 (pump #1) to withdraw fluid from a fluid source (e.g., a patient), the third pump 568 (pump #3) can withdraw fluid through the connector 570, thus allowing the infusion pump 518 to continue pumping normally as if the fluid path was not blocked by the infusion valve 521. If the measurement cycle is about two minutes long, this withdrawal by the third pump 568 can continue for approximately two minutes. Once the infusion valve 521 is open again, the third pump 568 (pump #3) can reverse and insert the saline back into the system at a low flow rate. Preferably, the time between measurement cycles is longer than the measurement cycle itself (e.g., longer than two minutes). Accordingly, the third pump 568 can insert fluid back into the system at a lower rate than it withdrew that fluid. This can help prevent an alarm by the infusion pump.

One of skill in the art can appreciate that the centrifuge and measurement process depicted in 550 can be replaced by other measurement methodologies to include indwelling biosensor technologies, optical measurement technologies that do not require centrifugation and in somatic strip methodologies were the sample is simply placed on the measurement system.

The present invention has been described in connection with various example embodiments. It will be understood that the above description is merely illustrative of the applications of the principles of the present invention, the scope of which is to be determined by the claims viewed in light of the specification. Other variants and modifications of the invention will be apparent to those of skill in the art.

What is claimed is:

1. A method of determining the patency of a blood access site in use with a blood access system, comprising:
a. Operating the blood access system and determining one or more characteristics of pressure, flow rate, or both, of fluid flow within the blood access system at a plurality of times;
b. Determining from characteristics whether the blood access site is suitable for continued access.

2. A method of managing a blood access site in use with a blood access system, comprising:
a. Determining the patency of the blood access site at a plurality of times, each time according to the method of claim 1;
b. If the determined patency indicates patency decreasing to a value below a predetermined value, or at a rate greater than a predetermined rate, then operating the blood access system to generate access site cleaning actions that enhance patency of the blood access site.

3. A method of managing a blood access site in use with a blood access system, comprising:
a. Determining the patency of the blood access site at a plurality of times, each time according to the method of claim 1;
b. If the determined patency indicates patency decreasing to a value below a predetermined value, or at a rate greater than a predetermined rate, then communicating an alert signal to medical personnel.

4. A method as in claim 1, wherein step b comprises comparing the determined characteristics to a range of characteristics defined for the blood access system, and if the determined characteristics are not within the range then indicating that the blood access site is not suitable for continued access.

5. A method as in claim 1, wherein step b comprises comparing the determined characteristics to a range of characteristics determined from characteristics determined previously for the same patient, and if the determined characteristics are not within the range then indicating that the blood access site is not suitable for continued access.

6. A method as in claim 1, wherein the blood access system comprises one or more of the blood access systems described in the specification.

7. A method as in claim 1, wherein the determined characteristics comprise:
a. “pressure one”, defined as the minimum sustained flow rate achieved at a predetermined pressure;
b. “pressure two”, defined as the change in pressure one over a plurality of blood access events.

8. A method as in claim 1, wherein the determined characteristics comprise:
a. “flow one”, defined as the pressure required to transport fluid through the access site at a predetermined flow rate;
b. “flow two”, defined as the change in flow one over a plurality of blood access events.

9. A method as in claim 1, wherein the determined characteristics comprise:
a. “decay one”, defined as the decay of pressure when flow is stopped;
b. “decay two”, defined as the change in decay one over a plurality of blood access events;
c. “growth one”, defined as the increase in pressure when flow is started or increased;
d. “growth two”, defined as the change in growth one over a plurality of blood access events.

10. A method as in claim 1, wherein step b) comprises comparing the determined characteristics to a range of characteristics determined from characteristics determined previously for other patients, and if the determined characteristics are not within the range then indicating that the blood access site is not suitable for continued access.

11. A method as in claim 10, wherein the other patients are patients with characteristics similar to the present patient.

12. A method as in claim 11, where the previous patients are patients with similar blood hematocrit as the present patient.

13. A method of automatically cleaning a blood access system, comprising:
a. Configuring the system such that a cleaning fluid can be transported through it without substantial infusion into the patient; and
b. Transporting a cleaning fluid through at least a portion of the system with variable fluid flow rates, or variable fluid flow directions, or both.

14. A method of determining that an occlusion in a blood access system is likely to occur, comprising:
a. Operating the blood access system and sensing pressure or flow at two or more places in the system;
b. Determining the likelihood of an occlusion from the relationship between the two or more sensed values.

15. A method of determining that an occlusion is present in a blood access system having bidirectional flow, comprising:
a. Operating the blood access system and sensing pressure or flow in at least one place in the system, wherein the pressure is at the connection to the patient, system controls flow rate and measures pressure, or pulls at a pump pressure and monitors pressure at the patient;
b. Determining that an occlusion is present when the pressure exceeds a threshold.

16. A method of automatically managing a blood access system in the presence of an actual or predicted occlusion, comprising:
a. Detecting that an occlusion is present or that an occlusion is likely to occur;
b. Controlling pressure or flow to avoid overpressure at patient, reverse flow rate, clean the system, or take residual blood to waste.
